(19) United States
(12) Patent Application Publication
(10) Pub. No.: US 2011/0034345 A1
(43) Pub. Date: Feb. 10, 2011

(54) METHODS FOR DIAGNOSIS OF MACULOPATHIES

(75) Inventors: Itay Chowers, Moshav Beit Zait (IL); Avraham Weiss, Gush Etzion (IL); Michal Lederman, Jerusalem (IL)

Correspondence Address: OLIFF & BERRIDGE, PLC, P.O. BOX 320850, ALEXANDRIA, VA, 22320-4850, US

(73) Assignee: Hadasit Medical Research Services & Development Limited, Jerusalem (IL)

(21) Appl. No.: 12/531,179

(22) PCT Filed: Mar. 13, 2008
(86) PCT No.: PCT/IL08/00361

§ 371 Date: Jan. 25, 2010

Related U.S. Application Data
(60) Provisional application No. 60/906,857, filed on Mar. 14, 2007.

(51) Int. Cl. C40B 30/04; C07H 21/04; C07K 16/00; C12Q 1/68; C40B 40/06

(52) U.S. Cl. 506/9; 536/24.3; 536/24.33; 530/387.1; 435/6; 506/16

(57) ABSTRACT

The present disclosure provides methods for diagnosing a maculopathy. Specifically, the methods are based on determination of a level of at least one biological marker of a maculopathy in a bodily fluid sample of an individual (e.g. blood sample) and comparing the level of the assayed biological marker with the level of prior determined cut off standards. The level of the biological marker provides information regarding the state of the individual, such as whether the individual has the assayed maculopathy, is predisposed to develop said maculopathy, is responsive to treatment, and others.

### FIELD OF THE INVENTION

This invention relates to diagnosis as well as to prognosis of maculopathies.

### BACKGROUND OF THE INVENTION

The retina is a thin layer of light-sensitive tissue that lines the inside wall of the back of the eye. When light enters the eye, the cornea and lens focus the light onto the retina, the transparent, light-sensitive membrane on the inner surface of the back of the eye. The central area of the retina, i.e. the macula, primarily contains a high density of color-sensitive photoreceptor cells. These cells, called cones, produce the sharpest visual images and are responsible for central vision. The peripheral area of the retina, which surrounds the macula, contains mainly photoreceptor cells called rods, which respond to lower lighting levels but are not color sensitive. The rods are responsible for peripheral vision and night vision.

The optic nerve carries signals generated by the photoreceptors (cones and rods). Each photoreceptor sends a tiny branch to join the optic nerve. The optic nerve extends into the brain and connects to neurons that carry signals to the vision center of the brain, where they are interpreted as visual images.

The optic nerve and the retina have a rich supply of blood vessels that carry blood and oxygen. Part of this supply of blood vessels comes from the choroid, which is the layer of blood vessels that lies between the retina and the outer white coat of the eye (the sclera). The central retinal artery (the other major source of blood to the retina) reaches the retina near the optic nerve and then branches out within the retina.

Various retinal disorders and diseases involve abnormalities in any one of the components of the retina which may lead to blindness. The most common cause of vision loss and blindness in developed countries is age related macular degeneration (AMD).

AMD is characterized by two stages. The most common form of macular degeneration is the “dry” or non-neovascular stage of age related macular degeneration which is thought to result from oxidative injury and inflammation in the retina and choroid. A more severe form is termed “wet” or neovascular age related macular degeneration. In this form, blood vessels in the choroidal layer (a layer underneath the retina providing nourishment to the retina) break through a thin protective layer between the two tissues. These blood vessels may grow abnormally directly beneath the retina in a rapid uncontrolled fashion, resulting in bleeding, exudation, or eventually scar tissue formation in the macula which leads to severe loss of central vision. The neovascular (“wet”) stage of the disease develops in about 10-15% of individuals having dry AMD, and often leads to substantial visual loss.

It has been shown that treating AMD patients with oral supplements of antioxidant vitamins and zinc significantly reduce the risk of visual loss in these patients (A randomized, placebo-controlled, clinical trial of high-dose supplementation with vitamins C and E, beta carotene, and zinc for age-related macular degeneration and vision loss: AREDS report no, 8. Arch Ophthalmol 2001; 119:1417-36). Patients with the dry form of the disease may be periodically examined to enable early detection of conversion to the neovascular stage of the disease (Preferred practice pattern: Age related macular degeneration. American Academy of Ophthalmology, San Francisco. USA, 2003). However, unfortunately, most AMD patients are not diagnosed in the early stage of the disease and are first seen by an ophthalmologist only after they are symptomatic of the disease, i.e. only after the advanced stage of the disease has developed and substantial visual loss has occurred (Cervantes-Castaneda R A, Banin E, Hemo I, Shpigel M, Averbukh E, Chowers I. Lack of benefit of early awareness to age-related macular degeneration. Eye (2007 Jan. 12 [Epub ahead of print]). The patients who are not diagnosed early during the progress of AMD miss the potential benefits of vitamin therapy and periodic follow-ups.

Currently, AMD is diagnosed clinically by means of ophthalmoscopy. Studies have also shown increased oxidative products and anti-retinal antibodies in the blood of AMD patients (Gu X, Meer S G, Miyagi M, et al. Carboxyethylpyrrole protein adducts and autoantibodies, biomarkers for age-related macular degeneration. J Biol Chem 2003; 278:42027-35; Cherepanoff S, Mitchell P, Wang J J, Gillies M C. Retinal autoantibody profile in early age-related macular degeneration: preliminary findings from the Blue Mountains Eye Study. Clin Experiment Ophthalmol 2006; 34:590-5; Patel N, Ohbayashi M, Nugent A K, et al. Circulating anti-retinal antibodies as immune markers in age-related macular degeneration. Immunology 2005; 115:422-30). Yet, these factors are not utilized for diagnosis or screening of AMD. However, as also concluded by Cherepanoff et al. the retinal autoantibody profile in early AMD is complex, both in terms of antigenic targets and immunoglobulin isotypes and detection of potential disease-associated autoantibodies will require a larger sample size and full characterization of the retinal antigens involved.

Thus, there is a need in the art for a simple and accurate tool for determining AMD at early stages of the disease.

### SUMMARY OF INVENTION

The invention is based on the surprising discovery that elevated levels of several biological markers in white blood cells or their protein products in the blood of age-related macular degeneration (AMD) patients, as compared to non-AMD individuals, correlated the existence of AMD.

Thus, in accordance with a first aspect, there are disclosed at least three methods associated with diagnosing maculopathy.

Firstly, there is disclosed a method for of determining a maculopathy, the method comprising:
determining a level of at least one biological marker of said maculopathy in a bodily fluid sample of the individual; and
comparing said level of said at least one biological marker with the level of prior determined standards, at least one standard that correlates level of said biological marker with a healthy state and one or more standards that correlate level of said biological marker with the existence of maculopathy, wherein

a level of said biological marker having a statistically significant deviation from said prior determined standard for a healthy state is indicative that said individual has said maculopathy or that said individual is predisposed to develop said maculopathy;

wherein said biological marker is selected from:
(i) a nucleic acid molecule comprising a nucleic acid sequence as depicted in any one of sequences ID Nos. 1 to 22, a functional fragment, derivative or splice variant of same; or
(ii) an expression product of (i) or a molecule comprising a functional fragment of said expression product.

Further discloses is a method for determining severity of a maculopathy in an individual comprising:
determining a level of at least one biological marker of said maculopathy in a bodily fluid sample of the individual; and
comparing said level of said at least one biological marker with the level of prior determined standards that correlate level of said biological marker with severity of maculopathy;

wherein said biological marker is selected from:
(i) a nucleic acid molecule comprising a nucleic acid sequence as depicted in any one of SEQ ID Nos. 1 to 22, a functional fragment, derivative or splice variant of said nucleic acid sequence; or
(ii) an expression product of (i) or a molecule comprising a functional fragment of said expression product.

Yet, further discloses is a method for determining the effectiveness of a maculopathy therapeutic treatment of an individual, the treatment comprises administering a therapeutic agent to the individual, the method comprises determining the level of at least one biological marker of said maculopathy in a bodily fluid sample obtained from said individual, in two or more successive time points, one or more of which is during therapeutic treatment, wherein a difference in the level being indicative of effectiveness of the therapeutic treatment;

wherein said biological marker is selected from:
(a) a nucleic acid molecule comprising a nucleic acid sequence as depicted in any one of SEQ ID Nos. 1 to 22, a functional fragment, derivative or splice variant of said nucleic acid sequence; or
(b) an expression product of (i) or a molecule comprising a functional fragment of said expression product.

When determining effectiveness of treatment, the latter may include a variety of therapeutic modalities which are to date utilized to treat maculopathies. For example, for AMD such therapies may include, without being limited thereto, administration of anti vascular endothelial growth factor (VEGF) compounds such as Bevacizumab (Avastin) or Ranibizumab (Lucentis), by intravitreal, intravenous, or other route, treatment with photodynamic therapy (PDT), oral supplementations of vitamins and minerals, or other novel treatments which my be utilized to treat AMD in the future.

In accordance with another aspect, there is disclosed a nucleic acid probe for use in determining in an individual a state of maculopathy or a predisposition to develop said maculopathy, the probe being at least 80% complementary with a nucleic acid molecule comprising a sequence disclosed in SEQ ID NOs. 1 to 22, or with a fragment, derivative or splice variant of a sequence from SEQ ID NOs. 1 to 22.

In accordance with yet another aspect, there is disclosed an oligonucleotide primer pair for use in determining in an individual a state of maculopathy, a predisposition to develop said maculopathy, said primer pair being at least 80% complementary with a portion of a nucleic acid molecule comprising a sequence disclosed in SEQ ID NOs. 1 to 22, or with a fragment, derivative or splice variant of the sequence from SEQ ID NOs. 1 to 22.

Another aspect in accordance with the present disclosure provides a nucleic acid array comprising one or more probes as disclosed herein.

Yet, another aspect provides antibody capable of binding to a biological marker within a bodily fluid sample of an individual, if present in the sample, the biological marker selected from:

(i) a nucleic acid molecule comprising a nucleic acid sequence as depicted in any one of SEQ ID Nos. 1 to 22, a functional fragment, derivative or splice variant of said nucleic acid sequence; or

(ii) an expression product of (i) or a molecule comprising a functional fragment of said expression product

Finally, disclosed herein is a test kit for use in determining in an individual a state of maculopathy, or whether an individual is in predisposition to develop said maculopathy the test kit comprising one or more probes, or one or more primer pairs, or a combination of both, or an antibody all being as disclosed herein.

### DETAILED DESCRIPTION OF FIGURES

In order to understand the invention and to see how it may be carried out in practice, embodiments will now be described, by way of non-limiting example only, with reference to the accompanying drawings, in which:

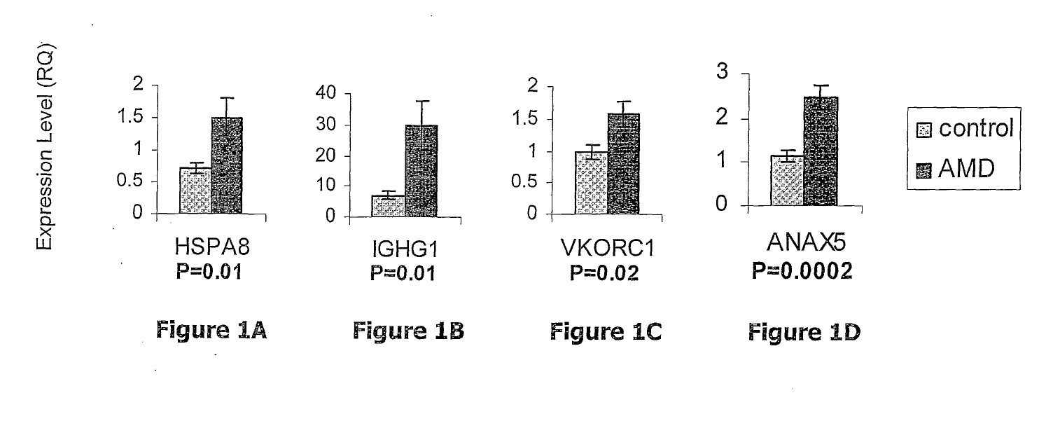

FIGS. 1A-1D are bar graphs showing the average (SE) mRNA levels of 4 genes in white blood cells from neuvascular age related macular degeneration (NVAMD) patients and controls according to real time quantitative-PCR (QPCR). Ratios of average mRNA levels in patients vs. controls: HSPA8-2.1, IGHG1-4.3, VKORC1-1.6, ANXA5-2.1.

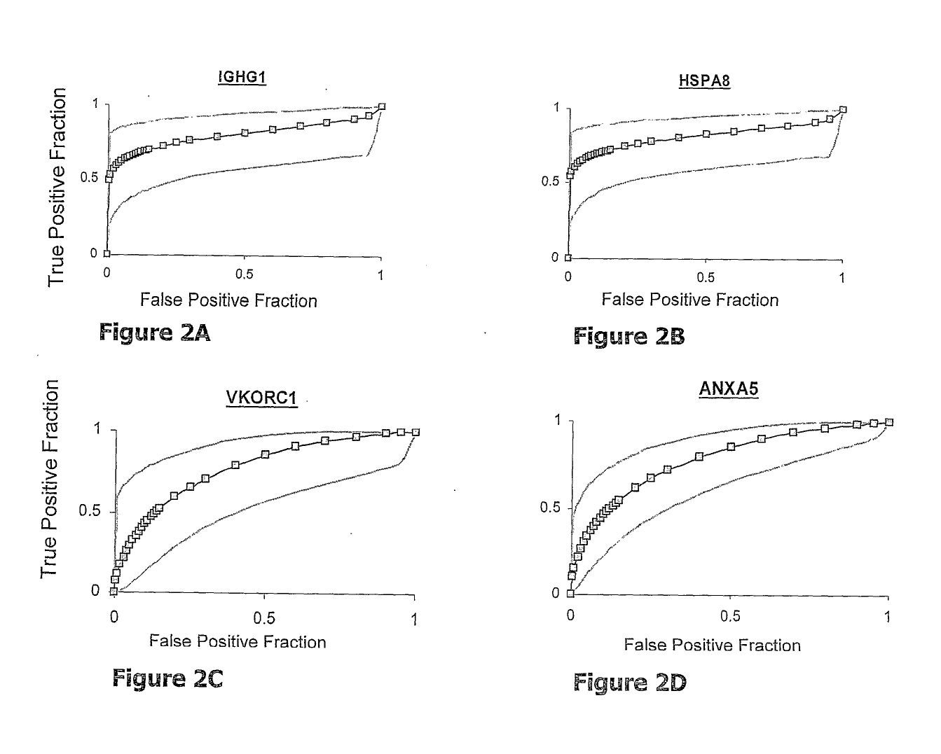

FIGS. 2A-2D are receiver operator curves (ROC) characteristics based on QPCR results showing the true positive rate against the false positive rate for different possible cut off points. Corresponding areas under curve were: IGHG1=0.803, HSPA8=0.815, VKORC1-0.776, and ANXA5-0.781.

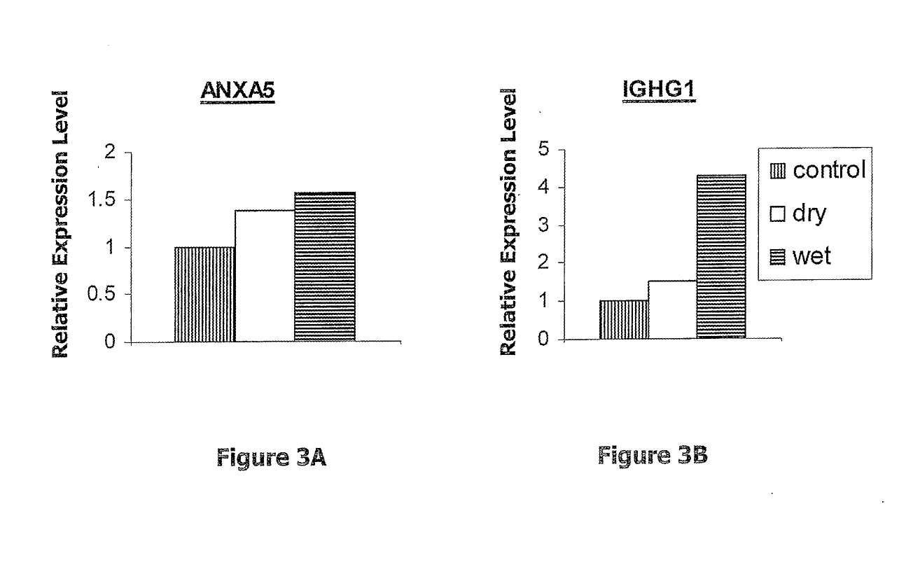

FIGS. 3A-3B are bar graphs showing a trend towards higher expression levels of ANXA5 (FIG. 3A) and IGHG1 (FIG. 3B) in dry (non-neovascular) AMD patients (n=10) vs. controls (n=29), and in wet AMD patients (n=26) compared with dry AMD patients.

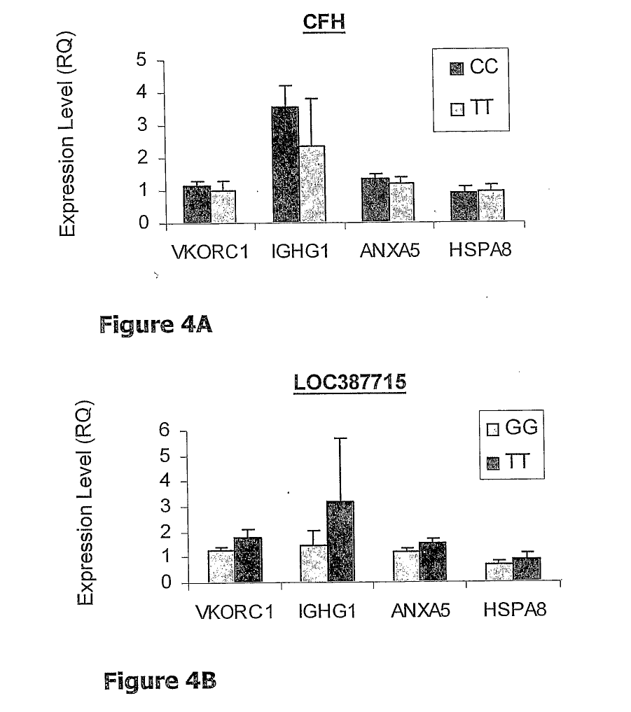

FIGS. 4A-4B are bar graphs showing correlation between genotypes for rs1061170 Single Nucleotide Polymorphism (SNP) in Complement Factor H (CFH) (FIG. 3A) and LOC387715 SNP (FIG. 3B) and gene expression level, using average expression levels (SE) of 4 genes according to QPCR. For rs1061170 SNP in CFH (C=risk allele, T=wild type allele), and for LOC387715 SNP (T=risk allele, G=wild type allele). P=none significant for all genes and both polymorphisms.

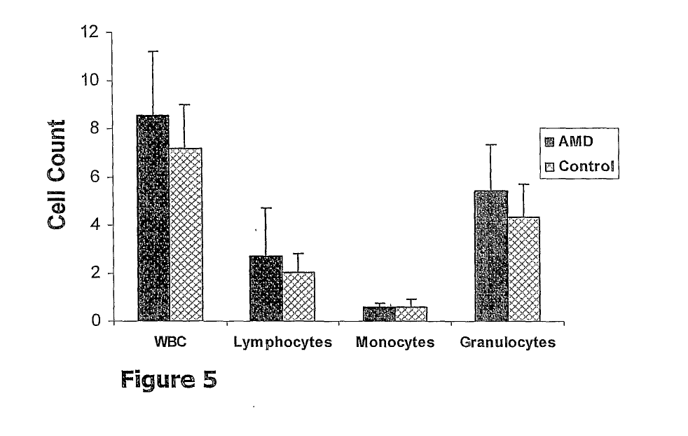

FIG. 5 is a bar graph showing a comparison of the average (SE) number of white blood cells (WBC), lymphocytes, monocytes and granulocytes in samples from NVAMD patients and controls. P=none significant for each comparison.

### DESCRIPTION OF THE INVENTION

The present invention provides methods and tools for diagnosing maculopathy (e.g. AMD). The novel methods involve performing a relatively simple test on a bodily fluid, such as a blood test.

The invention is based on a research which involved microarray analysis and real time quantitative polymerase chain reaction (quantitative RT-PCR-QPCR), which led to the identification of a group of genes exhibiting altered expression in white blood cells of patients with age-related macular degeneration (AMD). The representative mRNAs of the identified genes are provided in Table 1 and in the Sequence Listing forming part of this application). Specifically, the measurement of levels of these genes at the mRNA level using RT-QPCR or at the protein level in the blood plasma or serum demonstrated increased levels in both the dry and wet stages of AMD and it was envisaged by the inventors that these mRNA may serve as bio-markers for AMD and similar maculopathies to facilitate their diagnosis, including at an early stage.

Thus, disclosed herein is a method of determining if an individual has a maculopathy or is in a predisposition to develop said maculopathy, the method comprising:
(a) determining a level of at least one biological marker of said maculopathy in a bodily fluid sample of the individual; and
(b) comparing the level of said at least one biological marker with a the level of prior determined standards, at least one standard that correlates level of said biological marker with a healthy state and one or more standards that correlate level of said biological marker with the existence of maculopathy, wherein;

a level of said biological marker having a deviation from a prior determined cut off standard value for a healthy state is indicative that said individual has maculopathy or that said individual is predisposed to develop said maculopathy;

wherein said biological marker is selected from:
(i) a nucleic acid molecule comprising a nucleic acid sequence as depicted in any one of Sequence Identification (SEQ ID) Nos. 1 to 22, a functional fragment, derivative or splice variant of said nucleic acid sequence; or
(ii) an expression product of (i), or a molecule comprising a functional fragment of said expression product.

According to a preferred embodiment, there is disclosed a method for determining whether an individual has AMD or is in a predisposition to develop AMD, the method comprising:
(a) determining a level of a biological marker of said AMD in a bodily fluid sample of the individual; and
(b) comparing said level of said biological marker with the level of prior determined standards, at least one standard that correlates level of said biological marker with a healthy state and one or more standards that correlate level of said biological marker with the existence of maculopathy, wherein a level of said biological marker is higher than a cut off standard value for a healthy state is indicative that said individual has said AMD or that said individual is predisposed to develop said AMD;

wherein said biological marker is selected from:
(1) a nucleic acid molecule comprising a nucleic acid sequence as depicted in any one of SEQ ID Nos. 1 to 22, a functional fragment, derivative or splice variant of said nucleic acid sequence; or
(ii) an expression product of (i) or a molecule comprising a functional fragment of said expression product.

Further, disclosed herein is a method for determining severity of a maculopathy in an individual comprising:
(a) determining a level of at least one biological marker of said maculopathy in a bodily fluid sample of the individual; and
(b) comparing said level of said at least one biological marker with the level of prior determined standards that correlate level of said biological marker with the severity of maculopathy;

wherein said biological marker is selected from:
(i) a nucleic acid molecule comprising a nucleic acid sequence as depicted in any one of SEQ ID Nos. 1 to 22, a functional fragment, derivative or splice variant of said nucleic acid sequence; or
(ii) an expression product of (i) or a molecule comprising a functional fragment of said expression product.

Thus, in accordance with the methods disclosed herein it is possible not only to determine whether an individual has a maculopathy, but also the severity of the condition. This provides the practitioner (e.g. the physician) with means for determining the type of treatment to be given to the individual, as well as for assessing the chances that the individual will respond to a specific treatment or, in certain circumstances, be considered a non-responder to one or another particular treatment.

In a further embodiment, there is disclosed a method for determining the effectiveness of a maculopathy therapeutic treatment of an individual, the treatment comprises administering a therapeutic agent to the individual, the method comprises determining the level of at least one biological marker of said maculopathy in a bodily fluid samples obtained from said individual, in two or more successive time points, one or more of which is during therapeutic treatment, wherein a difference in the level being indicative of effectiveness of the therapeutic treatment;

wherein said biological marker is selected from:
(i) a nucleic acid molecule comprising a nucleic acid sequence as depicted in any one of SEQ ID Nos. 1 to 22, a functional fragment, derivative or splice variant of said nucleic acid sequence; or
(ii) an expression product of a nucleic acid molecule comprising a nucleic acid sequence as depicted in any one of SEQ ID Nos. 1 to 22, or a functional fragment of said expression product.

In accordance with this embodiment, one or more first samples are taken at a time point prior to initiation of the therapeutic treatment and one or more second samples are taken at a time point during the treatment, wherein a decrease in the level of the level determined in at least one said second samples as compared to that determined for at least one of said first samples is indicative that treatment is effective.

Alternatively, one or more first samples are taken at a time point during the treatment and one or more second samples are taken at a time point during the treatment subsequent to the time point of the one or more said first samples, such that a decrease in the level of the biological marker in one or more second samples as compared to the one or more first samples is indicative that treatment is effective.

Further alternatively, one or more first samples are taken at a time point during the treatment and one or more second samples are taken at a time point after the treatment has been discontinued, wherein an decrease in the level of the biological marker in the one or more second samples as compared to the one or more first samples is indicative that the treatment is effective.

In connection with the above, it is noted that an increased level of the biological marker or even no change in the level of the biological marker is considered indicative that the maculopathy is still active. In other words, that the treatment was ineffective, or not sufficiently effective so as to arrest the progression of the disease.

Further, it is noted that based on the level of the biological marker in the one or more second samples, the practitioner (e.g. the physician) may determine whether the individual requires one or more additional or alternative treatment sessions. In other words, where a level of the biological marker is over a predetermined cut-off value, it may serve as a prognostic factor to evaluate outcome of a treatment or the requirement of multiple treatment sessions.

The methods disclosed herein have the advantage that only an easily attainable bodily fluid (e.g. liquid) sample is required for the specified determinations.

Thus, to summarize, using a simple test, e.g. a blood test, the methods disclosed herein may have several applications:
As a screening test for identification of individuals at risk for having a maculopathy such as AMD, such as individuals over the age of 60 years and individuals with a family history of AMD or a similar ocular disorder. Individuals showing increased likelihood for having AMD or an ocular disorder according to the blood test may then be referred for further evaluation by an ophthalmologist and receive therapeutic treatment;
In situations where ophthalmoscopy is equivocal and diagnosis of a maculopathy such as AMD cannot be made conclusively, the invention may provide additional information in support of the diagnosis or against it;
To assess risk for development of a maculopathy, e.g., AMD in individuals, e.g. even in individuals with a normal ophthalmoscopy. The methods may facilitate early diagnosis and management of such diseases in asymptomatic individuals.

To assess risk for transition from the dry to the wet stage of the disease, e.g. AMD, in individuals who are diagnosed with the dry stage of the disease, thereby, facilitating scheduling of follow-up visits, and early diagnosis of transition to wet AMD.
To predict response to treatment in individuals who are diagnosed with wet AMD, the test may show which individual will respond to the therapy.

Thus, the present disclosure may be applied on large number of individuals. For example, for AMD which is a very common disorder with potential serious visual consequences, early detection of the disease may improve the outcome of such patients by facilitating appropriate follow up scheduling and by commencement of oral supplement of vitamins and minerals according to the AREDS study recommendations (A randomized, placebo-controlled, clinical trial of high-dose supplementation with vitamins C and E, beta carotene, and zinc for age-related macular degeneration and vision loss: AREDS report no. 8, Archives of Ophthalmology, 2001; 119: 1417-36).

For similar reasons, the invention may be applied on large number of individuals having other maculopathies, as appreciated by those versed in the art of ophthalmology.

All the above methods are applicable with respect to the determination of the existence and/or condition of a maculopathy. A “maculopathy” in the context of the present disclosure includes any macular disease leading to degeneration of retina and/or retinal pigment epithelium and/or choroid in the macula area, and/or choroidal neovascularization. Examples for such diseases are age related macular degeneration, myopic maculopathy, pattern dystrophy, and any other cause of choroidal neovascularization. A preferred embodiment concerns maculopathy associated with macular damage or degeneration or with choroidal neovascularization; a more preferred embodiment concerns AMD.

The existence of a maculopathy is determined as well as the predisposition of the individual to develop maculopathy. Predisposition denotes the tendency of the individual to develop (or to have a higher risk of developing) a maculopathy, without detectable symptoms thereof, namely, in pre-symptomatic or pre-diseased individuals.

Reverting to the methods disclosed herein, the level of the biological marker may be determined by quantitative as well as qualitative measuring, Both qualitative and quantitative determination methods can be used for diagnostic, prognostic and therapy planning purposes, as will be further discussed below. A level considered to be higher than a previously determined level is indicative that the disease is active, while, similarly, a decrease in the level as compared to a previously measured level is indicative of an improvement in the condition of the treated individual, namely, that the treatment is effective.

In connection with the examined individual, it is generally noted that the term “individual” is not limited to a human being but may also be other organisms including but not limited to mammals, plants, bacteria, or cells derived from any of the above.

The “biological marker” in the context of the present disclosure includes any molecule in the form of a nucleic acid sequence (thus referred to at times by the term “nucleic acid-based biological marker”) or amino acid sequence (thus referred to at times by the term “expression product” or “amino acid-based biological marker”), which is a characteristic trait of one or more conditions being encompassed by the broad term “maculopathy”, the biological marker thus facilitates diagnosis of a maculopathy as well as differential diagnosis of one condition from other, similar macular conditions.

When referring to a nucleic acid based biological marker, the sequence may be a mRNA comprising or having the sequence depicted in any one of SEQ ID NOs:1-22 (generally referred to herein by the term “original nucleic acid molecule”), a fragment of said sequence, a derivative of said sequence as well as a splice variant of said sequence.

The nucleic acids sequences depicted in the Sequence Listing include:

| | |
|---|---|
| CCNB1; | (Accession No. NM_031966, |
| | SEQ ID NO. 1) |
| ANXA5; | (Accession No. NM_001154.2, |
| | SEQ ID NO. 2) |
| CSE1L; | (Accession No. NM_001316.2, |
| | SEQ ID NO. 3) |
| EIF5A; | (Accession No. NM_001970.3, |
| | SEQ ID NO. 4) |
| TPD52; | (Accession No. NM_005079.2, |
| | SEQ ID NO. 5) |
| LOC441050; | (Accession No. XM_496721.3, |
| | SEQ ID NO. 6) |
| FANCG; | (Accession No. NM_004629.1, |
| | SEQ ID NO. 7) |
| HLA-DQA2; | (Accession No. NM_020056.2, |
| | SEQ ID NO. 8) |
| IGHG1; | (Accession No. NG_001019, |
| | SEQ ID NO. 9) |
| ISOC1; | (Accession No. NM_016048.2, |
| | SEQ ID NO. 10) |
| TBC1D7; | (Accession No. NM_016495.2, |
| | SEQ ID NO. 11) |
| NSUN2; | (Accession No. NM_017755.4, |
| | SEQ ID NO. 12) |
| ACN9; | (Accession No. BCO28409, |
| | SEQ ID NO. 13) |
| VKORC1; | (Accession No. NM_024006, |
| | SEQ ID NO. 14) |
| TXNDC5; | (Accession No. NM_030810, |
| | SEQ ID NO. 15) |
| RBBP4; | (Accession No. BC053904, |
| | SEQ ID NO. 16) |
| TUBA8; | (Accession No. NM_018943, |
| | SEQ ID NO. 17) |
| ZDHHC4; | (Accession No. NM_018106, |
| | SEQ ID NO. 18) |
| LP8165; | (Accession No. BC036520, |
| | SEQ ID NO. 19) |
| HSPA8 | (Accession No. NM_006597, |
| | SEQ ID NO. 20) |
| MYO5A; | (Accession No. NM_000259, |
| | SEQ ID NO. 21) |
| and | |
| IGHG2. | (Accession No. NG_001019, |
| | SEQ ID NO. 22) |

It is to be understood that in the context of the present disclosure, the nucleic acid-based biological marker may have one of the above sequence, however, may also comprise a sequence comprising at least 80%, preferably at least about 90% to 95%, and more preferably from about 98 to 100%, homology with a sequence depicted in any one of sequences ID Nos. 1 to 22.

Further, in the context of the present disclosure, nucleic acid based biological marker may comprise a contiguous sequence of at least 20 nucleic acid residues having at least 80%, preferably at least about 90% to 95%, and more preferably from about 98 to 100%, homology with a sequence depicted in any one of sequences ID Nos. 1 to 22.

The term “homology” as used herein refers to the percentage of residues that are identical in two compared sequences when the sequences are optimally aligned.

In the context of the present disclosure, the term “mRNA” should be construed as including pre-mRNA transcript(s), transcript processing intermediates, mature mRNA(s) ready for translation and transcripts of the gene or genes, or nucleic acids derived from mRNA transcript(s). Transcript processing may include splicing, editing and degradation. Further, as used herein, a nucleic acid derived from an mRNA transcript refers to a nucleic acid for whose synthesis, the mRNA transcript or a subsequence thereof has ultimately served as a template. Thus, a cDNA reverse transcribed from a mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, mRNA transcribed from the cDNA, DNA amplified from the genes, RNA transcribed from amplified DNA, and the like.

The “nucleic acid derivative” as used herein, includes any nucleic acid molecule in which at least one nucleic acid residue has been replaced, inserted, deleted or chemically modified. When the derivative includes a non-naturally occurring nucleic acid residue, the latter will typically have at least some structural features in common with a naturally occurring nucleoside or nucleotide such that when incorporated into a nucleic acid sequence, it will allow hybridization with a naturally occurring nucleic acid sequence in solution. Typically, derivatives will denote replacing and/or modifying the base, the ribose or the phosphodiester moiety. The changes can be tailor made to stabilize or destabilize hybrid formation or enhance the specificity of hybridization with a complementary nucleic acid sequence as desired.

When referring to “expression product” it should be understood to include any amino acid molecule encoded by one of the nucleic acid sequence depicted in SEQ ID NOs. 1-22 (at times, referred to by the term “true expression products”), by a derivative of a nucleic acid sequence depicted in SEQ ID NOs. 1-22, by a fragment of nucleic acid sequence depicted in SEQ ID NOs. 1-22, or by a splice variant of a nucleic acid sequence depicted in SEQ ID NOs. 1-22.

In the context of the present disclosure, the term “expression product” should also be construed to include a derivative of the true expression products, a fragment of such true products as well as molecules comprising said expression product or fragment thereof. In this connection, when referring to a molecule comprising a functional fragment of said expression product, it encompasses molecules comprising as well as consisting of said fragment of an expression product.

When referring to the level of the expression product it should be understood that level of the expression product or the level of activity of the expression product is determined.

A derivative of an expression product should be understood to include any amino acid based molecule which is different from the true expression product (encoded by any one of the nucleic acid sequence depicted in SEQ ID NOs. 1-22 (namely, the original nucleic acid molecules) by one or more of the following: substitution, deletion, insertion or chemical modification of one or more amino acid residues as compared to the true expression product. Substitution may include replacement of one or more naturally occurring amino acids with another naturally occurring amino acid and/or with one or more non-naturally occurring amino acid; insertion may include the inclusion of one or more naturally occurring or non-naturally occurring amino acid residue.

Naturally occurring amino acid refers to a moiety found within a peptide and is represented by —NH—CHR—CO—, wherein R corresponds to the side chain of the 20 naturally appearing amino acids; while non-naturally occurring amino acid include peptidomimetic, or the D-amino acid counterpart of naturally occurring amino acids. Amino acid analogs are well known in the art; a large number of these analogs are commercially available. The replacement of an amino acid is preferably a conservative replacement. A conservative replacement in the context of the present disclosure refers to the replacement of an original amino acid present in the true expression product with a naturally or non-naturally occurring amino having similar steric properties.

In the context of the present disclosure, the so-called derivatives, fragments, splice variants and any other altered form of the original nucleic acid molecule or expression product are to maintain the characteristic trait of the original nucleic acid molecules and their true expression products. The characteristic trait being that the expression of at least one of said molecules in a subject having maculopathy is elevated (as determined by statistical tests) as compared to their expression in a healthy subject. Such molecules will be regarded in the above and below disclosure as functional entities which may be used as biological marker of a maculopathy.

The methods of the present disclosure utilize prior determined standards. The “level of prior determined standards” as used herein denotes a level that may be determined by any method of known in the art, e.g. by determining a level of a biological marker in a bodily fluid or liquid sample from a statistically meaningful group of subjects considered to be healthy by other medical parameters (other conventional parameters for determining the disease stage, such as ophthalmoscopy), the level being for example, an average of levels from said group) or a range. It is noted that a level of a biological marker in a healthy subject also encompasses a null, namely, where there is no detection of the screened biological marker (i.e. zero level). In this connection, a prior determined standard indicative of a healthy state will be zero level of the marker in a tested sample.

The level of the biological marker may be determined by any technique known in the art. The level may be determined qualitatively as well quantitatively, using suitable probes, primer bases as well as other tools commonly known in biological assays.

When referring to quantitative measurements, it may include determining the concentration of the marker in the tested sample using quantitative real time RT-PCR, northern blots, western blots, and ELISA. The level of the biological marker is detected from a tested sample and the biological marker is preferably an mRNA, a protein, or a peptide.

The tested sample may comprise any bodily fluid, preferably, liquid, including blood, urine, cerebrospinal fluid, tears, saliva, lavage fluid. A preferred tested sample in accordance with the present disclosure is a blood sample. As used herein a “blood sample” denotes whole blood, plasma or serum.

When the bodily fluid sample is a blood sample, it is preferably whole blood, more preferably a sample comprising white blood cells (WBC).

The level of the biological marker is compared to that of a prior determined standard, e.g. a prior determined cut off standard. The difference in the level of the biological marker in the tested sample as compared to a prior determined standard should be statistically significant. The term “statistically significant” is used herein to denote that there is statistical evidence, as determined by traditional/conventional statistical tests, for a difference between the measured level of a biological marker in the tested sample and the level of the prior determined standard(s). In accordance with the present disclosure a p-value is considered none significant if it is larger than 0.05.

The methods of the invention employ oligonucleotide complementary to or substantially complementary to a portion of the biological marker. Such oligonucleotide serve as probes, primers and primer pairs for hybridization and a nucleic acid based biological marker, if present in the tested sample and thereby facilitating its detection.

Thus, in accordance with the present disclosure there is also provided a nucleic acid probe comprising a nucleic acid sequence being at least 80% complementary with a nucleic acid molecule comprising a sequence disclosed in SEQ ID NOs. 1 to 22, or with a fragment, derivative or splice variant of a sequence from SEQ ID NOs. 1 to 22.

The detection is carried out by identification of hybridization complexes between the probe and the biological marker. The probe, in some embodiments, may be attached to a solid support or to a detectable label. The probe will generally be single stranded and will generally be between 10 and 100 nucleotides, preferably between 15-25 nucleic acid residues.

Yet, there is disclosed herein an oligonucleotide primer pair being at least 80% complementary with a portion of a nucleic acid molecule comprising a sequence disclosed in SEQ ID NOs. 1 to 22, or with a fragment, derivative or splice variant of the sequence from SEQ ID NOs. 1 to 22.

In the context of the present disclosure the “primer” is a single-stranded oligonucleotide capable of acting as a point of initiation for template-directed synthesis of a nucleic acid molecule. The synthesis is conducted under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer. In general, the primers are single-stranded, between 10 and 40 bases in length and hybridize to regions of the template sequence located between 50 and 2000 bases apart.

Further, in the context of the present disclosure the “primer pair” is a set of two primers, each of which can serve to prime template-directed polymerization by a polymerase or transcriptase, which primers hybridize to the opposite strands of a double stranded nucleic acid sequence (“template”) in such manner as to direct the polymerization (and amplification) of the double-stranded nucleic acid sequence located between regions of primer hybridization. Such a primer pair can be used in the well known polymerase chain reaction (PCR). The design of primers pairs is well known in the art and will depend upon the particular sequence to be amplified.

As used herein, the term “complementary” or “substantially complementary” denotes the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res. 12: 203 (1984), incorporated herein by reference.

The “hybridization conditions” will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Hybridizations are usually performed under stringent conditions, for example, at a salt concentration of no more than 1 M and a temperature of at least 25° C. For stringent conditions, see, for example, Sambrook, Fritsche and Maniatis. “Molecular Cloning A laborato7y Manual”2nd Ed. Cold Spring Harbor Press (1989) which is hereby incorporated by reference in its entirety for all purposes above.

Suitable nucleic acids for preparing the oligonucleotide probes, primer as well as primer pairs may be selected from naturally occurring nucleic acids such as adenine, cytosine, guanine, uracil, and thymine.

Alternatively, non-naturally occurring or synthetic nucleic acids may be used to practice the methods disclosed herein. Examples of such nucleic acids include but are not limited to 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylrhoseudouridine, β-D-galactosylqueosine, T-Omethylguanosine, inosine, N⁶-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N⁶-methyladenosine, 7-methylguanosine, 5-methylaminomethyluridine, 5-methoxyaminomethyl-2-thiouridine, β-D-mannosylqueosine, 5-methoxycarbonylmethyluridine, 5-methoxyuridine, 2-methylthio-N⁶-isopentenyladenosine, N-((9-β-D-ribofuranosyl-2-methylthiopurine-6-yl)carbamoyl)threonine, N-((9-β-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-β-D-ribofuranosylpurine-6-yl) carbamoyl) threonine, 2′-O-methyl-5-methyluridine, 2′-O-methyluridine, wybutosine, and 3-(3-amino-3-carboxypropyl) uridine, 1-(2′-Deoxy-β-D-ribofuranosyl)-3-nitropyrrole. The probe or primer may also include protein nucleic acids (PNA).

The present disclosure specifically relates to PCR techniques (See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and each of which is incorporated herein by reference in their entireties for all purposes. The sample may be amplified on an array, i.e. when the probe is part of an array of probes each present in a known location on a solid support.

According to one embodiment, quantitative real time RT-PCR (QPCR) is utilized to measure mRNA levels in bodily fluid sample, e.g. white blood cells extracted from peripheral blood sample. QPCR is conducted by using commercially available assays (such as TaqMan®Gene Expression Assays from Applied Biosystems), or by using specific primers for the biomarker gene along with addition of Syber Green to the PCR reaction mixture. Results are normalized to the expression levels of an endogenous control gene such as glyceraldehyde-3-phosphate dehydrogenase (GAPDH) as detailed in the example sections below. (For reference regarding QPCR techniques please see: Arya M, et al.: Basic principles of real-time quantitative PCR. Expert Rev Mol Diagn. 2005; 5: 209-19)

The methods disclosed herein may also be applied for the detection of an amino acid based biological marker, namely, the detection of an expression product. To this end, the level of expression of the biological marker may be determined utilizing an antibody which binds specifically and/or selectively to the biological marker. Thus, antibodies also form part of the present invention.

It is to be understood that an antibody in the context of the present disclosure includes any of the IgG, IgM, IgD, IgA, and IgG antibodies, including, without being limited thereto, murine antibodies, humanized antibodies, human antibodies, polyclonal antibodies, monoclonal antibodies, chimeric antibodies, complementarity determining region (CDR)-grafted antibodies, antiidiotypic antibodies. An antibody refers to a whole antibody or fragments of the antibodies comprising the antigen-binding domain of the anti-variant product antibodies, e.g. scFv, Fab, F(ab′)₂, other antibodies without the Fc portion, biseptic antibodies, diabodies, single chain antibodies, other fragments consisting of essentially only the variable, antigen-binding domain of the antibody, etc.

Thus, there are disclosed herein also antibodies which bind to at least one biological marker of maculopathy, wherein said biological marker is an expression product of a nucleic acid molecule comprising a nucleic acid sequence as depicted in any one of SEQ ID Nos. 1 to 22, or a functional fragment or derivative of said expression product.

According to one embodiment, the antibody binds specifically (or selectively) to said biological marker.

The antibody will preferably be a monoclonal or polyclonal IgG directed towards the protein product (expression product) of the nucleic acid based biological marker.

Methods of preparing antibodies are well known in the art. Antibodies for application of the technique on protein products of genes described in Table #1 are commercially available, and will be purchased as required. For example, and as also detailed below, a mouse anti-Human ANXA5 monoclonal antibody which is appropriate for ELISA is available from Lifespan Biosciences and other companies. Similarly, a polyclonal Rabbit anti-Human CYCLIN B1 antibody which is appropriate for ELISA is available from Rockland Immunochemicals and other companies.

Methods for employing antibodies are well known in the art and include Western blot, Enzyme-Linked ImmunoSorbent Assay (ELISA), immunohistochemistry, immunoprecipitation.

In accordance with a further aspect, there is provided a test kit for use in determining a state of maculopathy or a predisposition to develop said maculopathy. The state of maculopathy may include the determination of one of the following:
whether an individual has a maculopathy;
whether an individual is in predisposition to develop maculopathy;
severity of maculopathy in an individual diagnosed as having the same;
risk for progression of a maculopathy in an individual diagnosed as having early stages of the disease
effectiveness of a therapeutic treatment provided to an individual diagnosed as having a maculopathy,

The test kit comprising one or more probes of the invention or one or more primer pairs according to the invention or a combination of both and instructions for use of the probe or primer pair in accordance with the method of the invention.

The invention has been described in an illustrative manner, and it is to be understood that the terminology which has been used, is intended to be in the nature of words of description rather than of limitation. Obviously, many modifications and variations of the present invention are possible in light of the above teaching.

The invention will now be described by way of non-limiting examples. It is to be understood that these example are provided for the purpose of illustration only and should not be construed as limiting. It is therefore, to be understood that within the scope of the appended claims, the invention may be practiced otherwise than as specifically described hereinafter.

Finally, as used in the specification and claims, the forms “a”, “an” and “the” include singular as well as plural references unless the context clearly dictates otherwise. For example, the term “a biological marker” includes one or more of such markers.

Further, as used herein, the term “comprising” is intended to mean that the methods or composition includes the recited elements, but not excluding others. Similarly, “consisting essentially of” is used to define methods and compositions that include the recited elements but exclude other elements that may have an essential significance on the invention. “Consisting of” shall mean excluding more than trace elements of other elements. Embodiments defined by each of these transition terms are within the scope of this invention.

Further, all numerical values, e.g., concentration or dose or ranges thereof, are approximations which are varied (+) or (−) by up to 20%, at times by up to 10% of from the stated values. It is to be understood, even if not always explicitly stated that all numerical designations are preceded by the term “about”. It also is to be understood, although not always explicitly stated, that the reagents described herein are merely exemplary and that equivalents of such are known in the art.

### SOME NON-LIMITING SPECIFIC EXAMPLES

### Example 1

### (A) Microarray Analysis

### Methods and Materials

(i) White Blood Cell Separation and RNA extraction

Four ml of blood placed in tubes containing EDTA was used for white blood cells (WBC) separation. Eight ml of hypotonic lysis buffer [155 mM NH₄Cl (Gadot, Or Akiva, Israel), 10 mM CH₂O₃NH₃ (SIGMA-Aldrich, St. Louis, Mo., USA), 0.1 mM EDTA (LT. Baker, Philipsberg, N.J., USA) (pH 7.4)] was added to the blood, after which the sample was stored on ice for 10 minutes, followed by centrifugation at 2000 g at 4° C. for 10 minutes. Supernatant was discarded, and the previous stage was repeated. The pellet of white blood cells was re-suspended in 1 ml of TRI Reagent (SIGMA). Total RNA was then extracted according to the manufacturer's instructions. Possible ruminants of DNA were degraded using DNA-free (Ambion, Austin, Tex., USA), and RNA samples were purified using Rneasy MinElute Cleanup Kit (QIAGEN, Hilden, Germany). Samples were then stored at −80° C. until further use.

### (ii) Fluorescent Labeled Complementary DNA

Templates were prepared by incubating 20 μg of total RNA with 2 μg oligo dT primers (GE Healthcare, Buckinghamshire, UK) in 15 μl water, at 70° C. for 10 minutes. Six μl of 5× first strand buffer, 3 μl dTT, 2 μl Super-Script II Reverse Transcriptase (Invitrogen, Paisley, UK), 1.2 μl dNTPs (Bio Lab, Valkenswaard, Netherlands), aminoallyl-dUTP (SIGMA) and 1 μl of RNAguard (Amersham Biosciences, Piscataway, N.J., USA) were added to the reaction tube, and incubated at 42° C. for 2 hours. The reaction was stopped by adding 10 μl 1M NaOH (Bio lab) and 10 μl 0.5M EDTA (pH 8) and incubating at 65° C. for 15 minutes. After being cleansed in Microcon YM-30 columns, cDNA was dried by centrifugation in a speedvac. Fluorescent dye cy3 or cy5 suspended in 9 μl carbonate buffer (0.1M NaHCO₃, pH 8.6, SIGMA) was added to the dried cDNA and incubated at room temperature for 1 hour in the dark. Adding 35 μl Sodium Acetate (MERCK, Whitehouse Station, N.J., USA) terminated labeling. Leftover reagents were cleared using the PCR Purification kit (QIAGEN, Hilden, Germany). Labeling efficiency was measured by spectrophotometer.

(iii) Microarray Analysis

Microarray analysis was performed as recently described (Meir et al. Investigative Ophthalmology Visual Science 2007; 48:4890-6). Briefly, oligonucleotide (approximately 70 bp long) spotted microarrays containing 36,000 features, designed by Operon was used. The analysis included 32 microarrays (16 NVAMD samples and 16 controls). A reference sample design was applied. Sample (either NVAMD or control RNA was labeled with cy3 and reference RNA was labeled with cy5. Labeled cDNAs from patients/controls and reference samples were combined and dried by speedvac, and resuspended in 50 μl of binding buffer made of 2.5 ml DDW, 1.25 ml SSCX20, 1.25 ml Formamide (Fluka, St. Gallen, Switzerland), 50 μl 10% SDS (BDH Chemicals, Poole, UK), 2.5 μl 8 mg/ml tRNA (Borringen). Microarrays were incubated with BSA blocking buffer [0.6 gr BSA (Amresco, Solon, Ohio, USA), 600 μl 10% SDS, 15 ml SSCX20, 41.7 ml DDW] at 42° C. for 45 minutes, then briefly rinsed in DDW and dried. The mixture of cDNA was drizzled on a cover slide, immediately covered with a microarray, and flipped over. After securing into a hybridization chamber, the microarray was incubated in a 42° C. bath for 18-22 hours. Before scanning, arrays were washed once in 0.1% SDS, SSCX1, twice in 0.1% SDS, SSCX0.1, and once in SSCX0.1—each for 5 minutes. Finally, arrays were submerged in DDW and dried by centrifugation for 2 minutes at 1000 g. Microarrays were scanned by the Axon4000B laser scanner and images were processed using the Axon GenePix Pro 4.1 software.

Two distinct statistical algorithms were utilized to identify altered expression patterns: significance analysis for microarray (SAM), and linear models for microarray data (LIMMA).

### Results

SAM analysis of microarray data identified 8 and 168 genes with AMD associated expression pattern, at a False Discovery Rate (FDR) of 0% and 10%, respectively. Several of these genes are associated with inflammation according to their functional classification. LIMMA analysis identified 52 genes with AMD associated expression at FDR of 20%. Twenty of these 52 genes were also identified by SAM at FDR of 10%.

Table 1 provides names, symbols and accession numbers of twenty representative mRNA sequences. The table also provides details on two additional genes, HSPA8 and MYO5A, which were identified by SAM analysis only and were included since according to their known function these genes are likely to be involved in the pathogenesis of the disease.

QPCR on four genes included in Table 1 was performed using an independent sample set of patients and controls. Results of QPCR confirmed microarray findings for each of the four genes which were tested (p<0.05 for each gene, t-test, please see example B below for detailed description of QPCR).

TABLE 1
mRNA associated with AMD

| | | GenBank | | SEQ |
|---|---|---|---|---|
| Symbol | Name | Accession # | Unigene # | ID No. |
| CCNB1 | CYCLIN B1 | NM_031966 | Hs.23960 | 1 |
| ANXA5 | annexin 5 | NM_001154.2 | Hs.480653 | 2 |
| CSE1L | CHROMOSOME SEGREGATION 1- | NM_001316.2 | Hs.90073 | 3 |
| | LIKE | | | |
| EIF5A | EUKARYOTIC TRANSLATION | NM_001970.3 | Hs.534314 | 4 |
| | INITIATION FACTOR 5A; EIF5A | | | |
| TPD52 | D52 | NM_005079.2 | Hs.368433 | 5 |
| LOC441050 | similar to unactive progesterone | XM_496721.3 | Hs.568288 | 6 |
| | receptor, 23 kD | | | |
| FANCG | X-RAY REPAIR, COMPLEMENTING | NM_004629.1 | Hs.591084 | 7 |
| | DEFECTIVE, IN CHINESE HAMSTER, | | | |
| | 9; XRCC9 | | | |
| HLA-DQA2 | | NM_020056.2 | Hs.591798 | 8 |
| IGHG1 | IgG HEAVY CHAIN LOCUS | NG_001019 | Hs.510635 | 9 |
| ISOC1 | CGI-111 | NM_016048.2 | Hs.483296 | 10 |
| TBC1D7 | DKFZp686N2317 | NM_016495.2 | Hs.484678 | 11 |
| NSUN2 | FLJ20303 | NM_017755.4 | Hs.481526 | 12 |
| ACN9 | DC11 | BC028409 | Hs.592269 | 13 |
| VKORC1 | VITAMIN K EPOXIDE REDUCTASE | NM_024006 | Hs.324844 | 14 |
| | COMPLEX, SUBUNIT 1 | | | |
| TXNDC5 | ERP46 | NM_030810 | Hs.150837 | 15 |
| RBBP4 | RETINOBLASTOMA-BINDING | BC053904 | Hs.647652 | 16 |
| | PROTEIN 4; RBBP4 | | | |
| TUBA8 | TUBULIN, ALPHA-8 | NM_018943 | Hs.137400 | 17 |
| ZDHHC4 | | NM_018106 | Hs.5268 | 18 |
| LP8165 | KIAA0251 | BC036520 | Hs.370781 | 19 |
| HSPA8 | Homo sapiens heat shock 70 kDa | NM_006597 | Hs.180414 | 20 |
| | protein 8 | | | |
| MYO5A | Homo sapiens myosin VA | NM_000259 | Hs.21213 | 21 |
| IGHG2 | IgG HEAVY CHAIN LOCUS | NG_001019 | | 22 |

Table 1 shows, from left to right, respectively, the symbols, names and representative GenBank and Unigene accession numbers of twenty-two identified biological markers (mRNA) whose levels were correlated with AMD and their SEQ ID NO's as provided in the SEQUENCE LISTING forming part of this application.

Following are references for the statistical analysis methods:

Smyth, G. K., Limma: linear models for microarray data, in Bioinformatics and Computational Biology Solutions using R and Bioconductor, R. Gentleman and S. D. V. Carey, R. Irizarry, W. Huber, Editors. 2005, Springer: New York. p. 397-420.

Significance analysis of microarrays applied to the ionizing radiation response. Tusher, V G, Tibshirani, R, Chu, G. Proc Natl Acad Sci USA. 2001 Apr. 24; 98 (9):5116-21.

As evident from the results, AMD was found to be associated with altered expression level of several genes in WBCs according to microarray analysis and this was validated on an independent set of samples using quantitative real time RT-PCR (QPCR). Such genes have thus been determined to be candidates for involvement in the pathogenesis of AMD and may serve as biomarkers for the disease to facilitate detection of AMD using blood tests to measure expression level of the mRNA or protein products of these genes.

### (B) QPCR Analysis

### Methods and Materials

Results of microarray were validated using real time quantitative RT-PCR (QPCR) on a set of additional 36 AMD patients (26 with neovascular AMD and 10 with non-neovascular AMD) and 29 age and gender matched unaffected controls which were not tested by the arrays (independent sample set) (FIG. 1) RNA was extracted as detailed in (A) above.

Primers for QPCR were prepared by selecting a unique complementary sequence of 10-40 bases to the nucleic acid biological markers disclosed herein (SEQ ID NO:1-22) and which amplifies a 200-400 base fragment from the cDNA of the target gene. Specifically, in the context of the present disclosure, when the sample is a blood sample comprising white blood cells, cDNA is synthesized from 1 μg total RNA extracted from separated white blood cells (as described above) following conventional reverse transcription using anchored oligo dT primers as was previously described (Meir et al. Investigative Ophthalmology Visual Science 2007; 48:4890-6).

The expression levels of biological markers was then assessed in each sample using GAPDH mRNA levels as the endogenous control to which each sample is normalized. Reactions are performed using the SYBR Green technique and TaqMan techniques and the primers as specified in Table 2. Levels of GAPDH were used as endogenous control for normalization of the expression levels. In Table 2, ng cDNA denotes the amount of cDNA from a sample which was used for the QPCR reaction; Syber denotes that QPCR was performed using specific primers with addition of Syber green to the reaction mixture as outlined in the examples; TaqMan denotes that QPCR was performed using TaqMan assay purchased from Applied Biosystems.

TABLE 2
Primers and assays used for quantification of
mRNA levels

| | ng | | Tech- |
|---|---|---|---|
| Gene | cDNA | Primer | nique |
| ANXA5 | 2 | QT00079275(QuantiTect | SYBR |
| | | Primer Assay, QIAGEN) | |
| GAPDH | 0.5 | F- TAGCCAAATTCGTTGTCATACC | SYBR |
| | | R- CTGACTTCAACAGCGACACC | |
| HSPA8 | 15 | QT00030079(QuantiTect | SYBR |
| | | Primer Assay, QIAGEN) | |
| IGHG1 | 40 | Hs00378230_g1(TaqMan Assay- | TaqMan |
| | | on-Demand, Applied Biosystems) | |
| VKORC1 | 0.1 | F- TTCTGTCTACCTGGCCTGGATC | SYBR |
| | | R- CACGTTGATAGCATAGGTGGTGA | |

Each tube contained 10 μl PCR mix and 2.8 μl primers—for SYBR Green. TaqMan reactions were performed following the protocol supplied by the manufacturer (Applied Biosystems). Amounts of cDNA were calibrated of each primer (Table 1). A total volume of 20 μl was completed by DDW. Amplification is measured throughout 40 cycles of 60° C. for 15 seconds, followed by 95° C. for 15 seconds, Samples were prepared in triplicates, and calculations were performed on the average value. Real-Time PCR reactions were carried out using the ABI Prism 7000 SDS Software or 7900HT Fast Real-Time PCR system (Applied Biosystems). Results were assessed by receiver operating curve (ROC) analysis (Receiver-operating characteristic (ROC) plots: a fundamental evaluation tool in clinical medicine. Clin Chem. Zweig M H, Campbell G. 1993; 39:561-77).

### Results

Significant higher expression levels were detected among NVAMD patients compared with controls for each of the genes tested (p<0.05 in each case, FIG. 1). Receiver operating curve analysis demonstrated that detection of the relative expression levels of genes in WBCs with QPCR can distinct NVAMD patients from controls with high area under the curve values for the four genes tested suggesting that such measurements may serve as a diagnostic test for the disease (FIGS. 2A-2D).

As to determination of NVAMD, QPCR showed a trend towards higher expression levels of ANXA5 and IGHG1 in dry (non-neovascular) AMD patients (n=10) vs. controls (n=29), and in wet AMD patients (n=26) compared with dry AMD patients (FIG. 3A-3B). Larger number of patients with dry AMD is currently being evaluated to further characterize the expression profile of the biomarker genes in this stage of the disease.

Genotyping demonstrated that gene expression pattern in WBC was not associated with the major risk SNPs for AMD in Complement Factor H (CFH, rs1061170), or LOC387715 (rs10490924)/HTRA1 (rs11200638) (FIGS. 4A-4B, respectively) suggesting that these risk SNPs do not underlie the gene expression patterns which were identified.

Complete blood counts were similar between patients and controls teaching that AMD associated gene expression pattern in WBCs was not dependent on the number of WBCs among patients and controls (FIG. 5). Thus, it was determined by the inventors that gene expression pattern is an independent biomarker for AMD.

### Example 2

### Diagnosis

### (i) Primer Preparation

Primers for QPCR are prepared by selecting a unique complementary sequence of 10-40 bases to the nucleic acid biological markers disclosed herein (SEQ ID NO:1-22) and which amplifies a 200-400 base fragment from the cDNA of the target gene. Specifically, in the context of the present disclosure, when the sample is a blood sample comprising white blood cells, cDNA is synthesized from 1 μg total RNA extracted from separated white blood cells (as described above) following conventional reverse transcription using anchored oligo dT primers (for details on reverse transcription protocol please see: Meir et al. Investigative Ophthalmology Visual Science 2007; 48:4890-6).

The expression levels of biological markers is then assessed in each sample using GAPDH mRNA levels as the endogenous control to which each sample is normalized. Reactions are performed using the SYBR Green or TaqMan techniques as illustrated in Example 1B above. Amounts of cDNA are calibrated of each primer. A total volume of 20 μl is completed by DDW. Amplification is measured throughout 40 cycles of 60″ for 15 seconds, followed by 95° C. for 15 seconds. Samples are prepared in triplicates, and calculations are performed on the average value. Real-Time PCR reactions are carried out using the ABI Prism 7000 SDS Software or 7900HT Fast Real-Time PCR system (Applied Biosystems).

(ii) mRNA Level Determination

The normal (healthy state) range of mRNA levels for each of the neucleic acid based biological markers is defined. This is performed by extracting total RNA from white blood cells as described above followed by QPCR with the specific primers for the biological markers mentioned in Table 1 (SEQ ID NO:1-22).

Similarly, standards for expression product (e.g. protein) levels are established using ELISA for the protein product of the specified genes. Protein levels are tested in blood as well as other bodily fluids (please see below for more details on protein testing).

To perform a test of RNA level: Four cc of venous blood is drawn from an individual into an EDTA containing tube. Total RNA is extracted as mentioned above and RNA is subjected to cDNA synthesis and QPCR using specific primers as descried above. Levels of RNA is recorded and compared with the established standard. A cut-off value above which a test result is considered abnormal is determined for each gene based on the Receiver Operating Characteristic (ROC) curve of the particular gene. Such cut-off values are determined based on sensitivity and specificity by selecting a value which is closest to the (0-on X axis, 1-on Y axis) point in the ROC analysis. Selection of this value is based on identifying the point yielding the minimal value for (1−sensitivity)²+(1−specificity)². Alternatively, the cut-off value is selected by calculating the Youden index (J), where J is defined as the largest distance between the ROC curve and the line showing results obtained by chance alone. J is calculated as J=maximum {sensitivity+specificity−1}.

Combination of measurements of mRNA levels of several genes may be utilized to better establish the diagnosis. In such cases individual cut-off points for each gene are determined and results for each gene are obtained from the individual as described above. The number of such results which are abnormal (larger than the cut-off value) are then recorded. Cut-off for each combination of tests is then determined based on ROC calculation as defined above where ratio of the number of positive to negative tests will be analyzed using ROC. Cut-off value for this calculated combination will be selected based on the two methodologies mentioned above.

Protein level is measured in blood or bodily fluids when the protein based biological marker is expressed and secreted. A preferred method to quantify the protein is ELISA.

Antibodies to the protein products of the genes mentioned in Table 1 are commercially available enabling the development of a specific ELISA assay. For example, a mouse anti-Human ANXA5 monoclonal antibody which is appropriate for ELISA is available from Lifespan Biosciences and other companies. Similarly, a polyclonal Rabbit anti-Human CYCLIN B1 antibody which is appropriate for ELISA is available from Rockland Immunochemicals and other companies. To measure protein level, five cc of blood is drawn into a tube containing EDTA or heparin (depending on the specific antibody). White blood cells are extracted as described above for proteins which are not secreted, and ELISA is performed on protein sample from the isolated white blood cells. When measuring secreted proteins, the ELISA is performed on a serum or plasma sample. Combination of several biomarkers may also be performed by calculating combined ROC curves as described above for individual biomarker.

The biological markers as defined herein provide a sensitive, specific and positive predictive tool for diagnosing individuals with different stages of a disease (early, intermediate, or advance) as compared to control (healthy) individuals.

Measurements of the mRNA levels and protein levels and activities of these genes are assessed as a diagnostic tool for maculopathies such as AMD (non-neovascular or neovascular) as well as maculopathies other than AMD, such as choroidal neovascularization (CNV) unrelated to AMD (myopic CNV, idiopathic CNV, CNV associated with inflammatory retinal or choroidal disorders, or CNV associated with trauma, etc.), myopic maculopathy, pattern dystrophy, and other degenerative maculopathies. These measurements provide comprehensive evaluation for diagnosis and risk assessment of maculopathies, as well as a basis upon which the biochemical and/or molecular pathways involved in the pathogenesis of maculopathies may be elucidated.

The measurements of the expression of these genes are also performed in asymptomatic individuals with normal macula appearance in ophthalmoscopy.

1-20. (canceled)

21. A method of determining a maculopathy, the method comprising:
(a) determining a level of at least one biological marker of said maculopathy in a bodily fluid sample of the individual; and
(b) comparing said level of said at least one biological marker with the level of prior determined standards, at least one standard that correlates level of said biological marker with a healthy state and one or more standards that correlate level of said biological marker with the existence of maculopathy, wherein
a level of said biological marker having a deviation from said a prior determined standard for a healthy state is indicative that said individual has said maculopathy or that said individual is predisposed to develop said maculopathy;
wherein said biological marker is selected from:
(i) a nucleic acid molecule comprising a nucleic acid sequence as depicted in any one of sequences ID Nos. 1 to 22, a functional fragment, derivative or splice variant of same; or
(ii) an expression product of (i) or a molecule comprising a functional fragment of said expression product.

22. A method for determining severity of a maculopathy in an individual comprising:
(a) determining a level of at least one biological marker of said maculopathy in a bodily fluid sample of the individual; and
(b) comparing said level of said at least one biological marker with the level of prior determined standards that correlate level of said biological marker with severity of maculopathy;
wherein said biological marker is selected from:
(i) a nucleic acid molecule comprising a nucleic acid sequence as depicted in any one of SEQ ID Nos. 1 to 22, a functional fragment, derivative or splice variant of said nucleic acid sequence; or
(ii) an expression product of (i) or a molecule comprising a functional fragment of said expression product.

23. A method for determining the effectiveness of a maculopathy therapeutic treatment of an individual, the treatment comprises administering a therapeutic agent to the individual, the method comprises determining the level of at least one biological marker of said maculopathy in a bodily fluid sample obtained from said individual, in two or more successive time points, one or more of which is during therapeutic treatment, wherein a difference in the level being indicative of effectiveness of the therapeutic treatment;
wherein said biological marker is selected from:
(a) a nucleic acid molecule comprising a nucleic acid sequence as depicted in any one of SEQ ID Nos. 1 to 22, a functional fragment, derivative or splice variant of said nucleic acid sequence; or
(b) an expression product of (i) or a molecule comprising a functional fragment of said expression product.

24. The method of claim 23, wherein one or more first samples are taken at a time point prior to initiation of the therapeutic treatment, where a level of the biological marker over a predetermined cut off standard permitting prediction of an outcome of a treatment or multiple treatment sessions.

25. The method of claim 23, wherein one or more first samples are taken at a time point during the treatment and one or more second samples are taken at a time point during the treatment subsequent to the time point of the one or more said first samples, such that a decrease in the level of the biological marker in one or more second samples as compared to the one or more first samples is indicative that treatment is effective.

26. The method of claim 23, wherein one or more first samples are taken at a time point during the treatment and one or more second samples are taken at a time point after the treatment has been discontinued, wherein an decrease in the level of the biological marker the one or more second samples as compared to the one or more first samples is indicative that the treatment is effective.

27. The method of claim 21, wherein said maculopathy is associated with macular damage or degeneration or with choroidal neovascularization.

28. The method of claim 22, wherein said maculopathy is associated with macular damage or degeneration or with choroidal neovascularization.

29. The method of claim 23, wherein said maculopathy is associated with macular damage or degeneration or with choroidal neovascularization.

30. The method of claim 27, wherein said maculopathy is selected from age-related macular degeneration (AMD) of the non-neovascular or neovascular stage, myopic maculopathy, myopic choroidal neovascularization (CNV), idiopathic CNV, CNV associated with inflammatory retinal or choroidal disorders, pattern dystrophy, or CNV associated with trauma.

31. The method of claim 28, wherein said maculopathy is selected from age-related macular degeneration (AMD) of the non-neovascular or neovascular stage, myopic maculopathy, myopic choroidal neovascularization (CNV), idiopathic CNV, CNV associated with inflammatory retinal or choroidal disorders, pattern dystrophy, or CNV associated with trauma.

32. The method of claim 29, said maculopathy is selected from age-related macular degeneration (AMD) of the non-neovascular or neovascular stage, myopic maculopathy, myopic choroidal neovascularization (CNV), idiopathic CNV, CNV associated with inflammatory retinal or choroidal disorders, pattern dystrophy, or CNV associated with trauma.

33. The method of claim 30, wherein said maculopathy is AMD.

34. The method of claim 31, wherein said maculopathy is AMD.

35. The method of claim 32, wherein said maculopathy is AMD.

36. The method of claim 21, wherein the bodily sample is selected from the group consisting of blood, urine, cerebrospinal fluid, tears, saliva, and lavage fluid.

37. The method of claim 22, wherein the bodily sample is selected from the group consisting of blood, urine, cerebrospinal fluid, tears, saliva, and lavage fluid.

38. The method of claim 23, wherein the bodily sample is selected from the group consisting of blood, urine, cerebrospinal fluid, tears, saliva, and lavage fluid.

39. The method of claim 36, wherein the blood is whole blood comprising white blood cells (WBC) and the biological marker is an mRNA, a protein or a peptide.

40. The method of claim 37, wherein the blood is whole blood comprising white blood cells (WBC) and the biological marker is an mRNA, a protein or a peptide.

41. The method of claim 38, wherein the blood is whole blood comprising white blood cells (WBC) and the biological marker is an mRNA, a protein or a peptide.

42. A nucleic acid probe for use as an agent for determining in an individual a state of maculopathy or a predisposition to develop said maculopathy, the probe being at least 80% complementary with a nucleic acid molecule comprising a sequence disclosed in SEQ ID NOs. 1 to 22, or with a fragment, derivative or splice variant of a sequence from SEQ ID NOs. 1 to 22.

43. An oligonucleotide primer pair for use as an agent for determining in an individual a state of maculopathy, a predisposition to develop said maculopathy, said primer pair being at least 80% complementary with a portion of a nucleic acid molecule comprising a sequence disclosed in SEQ ID NOs. 1 to 22, or with a fragment, derivative or splice variant of the sequence from SEQ ID NOs. 1 to 22.

44. A nucleic acid array comprising one or more probes according to claim 42.

45. An antibody capable of binding to a biological marker within a bodily fluid sample of an individual, if present in the sample, the biological marker selected from:
(i) a nucleic acid molecule comprising a nucleic acid sequence as depicted in any one of SEQ ID Nos. 1 to 22, a functional fragment, derivative or splice variant of said nucleic acid sequence; or
(ii) an expression product of (i) or a molecule comprising a functional fragment of said expression product.

46. A test kit for use in determining in an individual a state of maculopathy, or whether an individual is in predisposition to develop said maculopathy the test kit comprising at least one component selected from one or more nucleic acid probes, one or more oligonucleotide primer pairs, or a combination of both, and an antibody according to claim 45, wherein:
a) said probe being at least 80% complementary with a nucleic acid molecule comprising a sequence disclosed in SEQ ID NOs. 1 to 22, or with a fragment, derivative or splice variant of a sequence from SEQ ID NOs. 1 to 22; and
b) said primer pair being at least 80% complementary with a portion of a nucleic acid molecule comprising a sequence disclosed in SEQ ID NOs. 1 to 22, or with a fragment, derivative or splice variant of the sequence from SEQ ID NOs. 1 to 22.


SEQUENCE LISTING (22 sequences)

SEQ ID NO: 1 - DNA, 2101 bp, Homo sapiens
acgaacaggccaataaggagggagcagtgcggggtttaaatctgaggctaggctggctcttctcggcgtgctgcggcggaacggctgttggtttctgctgggtgtaggtccttggctggtcgggcctccggtgttctgcttctccccgctgagctgctgcctggtgaagaggaagccatggcgctccgagtcaccaggaactcgaaaattaatgctgaaaataaggcgaagatcaacatggcaggcgcaaagcgcgttcctacggcccctgctgcaacctccaagcccggactgaggccaagaacagctcttggggacattggtaacaaagtcagtgaacaactgcaggccaaaatgcctatgaagaaggaagcaaaaccttcagctactggaaaagtcattgataaaaaactaccaaaacctcttgaaaaggtacctatgctggtgccagtgccagtgtctgagccagtgccagagccagaacctgagccagaacctgagcctgttaaagaagaaaaactttcgcctgagcctattttggttgatactgcctctccaagcccaatggaaacatctggatgtgcccctgcagaagaagacctgtgtcaggctttctctgatgtaattcttgcagtaaatgatgtggatgcagaagatggagctgatccaaacctttgtagtgaatatgtgaaagatatttatgcttatctgagacaacttgaggaagagcaagcagtcagaccaaaatacctactgggtcgggaagtcactggaaacatgagagccatcctaattgactggctagtacaggttcaaatgaaattcaggttgttgcaggagaccatgtacatgactgtctccattattgatcggttcatgcagaataattgtgtgcccaagaagatgctgcagctggttggtgtcactgccatgtttattgcaagcaaatatgaagaaatgtaccctccagaaattggtgactttgcttttgtgactgacaacacttatactaagcaccaaatcagacagatggaaatgaagattctaagagctttaaactttggtctgggtcggcctctacctttgcacttccttcggagagcatctaagattggagaggttgatgtcgagcaacatactttggccaaatacctgatggaactaactatgttggactatgacatggtgcactttcctccttctcaaattgcagcaggagctttttgcttagcactgaaaattctggataatggtgaatggacaccaactctacaacattacctgtcatatactgaagaatctcttcttccagttatgcagcacctggctaagaatgtagtcatggtaaatcaaggacttacaaagcacatgactgtcaagaacaagtatgccacatcgaagcatgctaagatcagcactctaccacagctgaattctgcactagttcaagatttagccaaggctgtggcaaaggtgtaacttgtaaacttgagttggagtactatatttacaaataaaattggcaccatgtgccatctgtacatattactgttgcatttacttttaataaagcttgtggccccttttacttttttatagcttaactaatttgaatgtggttacttcctactgtagggtagcggaaaagttgtcttaaaaggtatggtggggatatttttaaaaactccttttggtttacctggggatccaattgatgtatatgtttatatactgggttcttgttttatatacctggcttttactttattaatatgagttactgaaggtgatggaggtatttgaaaattttacttccataggacatactgcatgtaagccaagtcatggagaatctgctgcatagctctattttaaagtaaaagtctaccaccgaatccctagtccccctgttttctgtttcttcttgtgattgctgccataattctaagttatttacttttaccactatttaagttatcaactttagctagtatcttcaaactttcactttgaaaaatgagaattttatattctaagccagttttcattttggttttgtgttttggttaataaaacaatactcaaatacaaaaaaaaaaaa

SEQ ID NO: 2 - DNA, 1630 bp, Homo sapiens
agggccggggtggggcgctggcgtttccgttgcttggatcagtctaggtgcagctgcggatccttcagcgtctgcatctcggcgtcgccccgcgtaccgtcgcccggctctccgccgctctcccgggggttcggggcacttgggtcccacagtctggtcctgcttcaccttcccctgacctgagtagtcgccatggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaacgtgtcacggggaagagctccctgctgtgtgcctgcaccaccccactgccttccttcagcacctttagctgcatttgtatgccagtgcttaacacattgccttattcatactagcatgctcatgaccaacacatacacgtcatagaagaaaatagtggtgcttctttctgatctctagtggagatctctttgactgctgtagtactaaagtgtacttaatgttactaagtttaatgcctggccattttccatttatatatattttttaagaggctagagtgcttttagccttttttaaaaactccatttatattacatttgtaaccatgatactttaattagaagcttagccttgaaattgtgaactcttggaaatgttattagtgaagttcgcaactaaactaaacctgtaaaattatgatgattgtattcaaaagattaatgaaaaataaacatttctgtccccctgaattat

SEQ ID NO: 3 - DNA, 3579 bp, Homo sapiens
tcaggctcgctgtcgcgccattttgccggggtttgaatgtgaggcggagcggcggcaggagcgggtagtgccagctacggtccgcggctggggttccctcctccgtttctgtatccccacgagatcctatagcaatggaactcagcgatgcaaatctgcaaacactaacagaatatttaaagaaaacacttgatcctgatcctgccatccgacgtccagctgagaaatttcttgaatctgttgaaggaaatcagaattatccactgttgcttttgacattactggagaagtcccaggataatgttatcaaagtatgtgcttcagtaacattcaaaaactatattaaaaggaactggagaattgttgaagatgaaccaaacaaaatttgtgaagccgatcgagtggccattaaagccaacatagtgcacttgatgcttagcagcccagagcaaattcagaagcagttaagtgatgcaattagcattattggcagagaagattttccacagaaatggcctgacttgctgacagaaatggtgaatcgctttcagagtggagatttccatgttattaatggagtcctccgtacagcacattcattatttaaaagataccgtcatgaatttaagtcaaacgagttatggactgaaattaagcttgttctggatgcctttgctttgcctttgactaatctttttaaggccactattgaactctgcagtacccatgcaaatgatgcctctgccctgaggattctgttttcttccctgatcctgatctcaaaattgttctatagtttaaactttcaggatctccctgaattttttgaagataatatggaaacttggatgaataattttcatactctcttaacattggataataagcttttacaaactgatgatgaagaggaagccggcttattggagctcttaaaatcccagatttgtgataatgccgcactctatgcacaaaagtacgatgaagaattccagcgatacctgcctcgttttgttacagccatctggaatttactagttacaacgggtcaagaggttaaatatgatttgttggtaagtaatgcaattcaatttctggcttcagtttgtgagagacctcattataagaatctatttgaggaccagaacacgctgacaagtatctgtgaaaaggttattgtgcctaacatggaatttagagctgctgatgaagaagcatttgaagataattctgaggagtacataaggagagatttggaaggatctgatattgatactagacgcagggctgcttgtgatctggtacgaggattatgcaagttttttgagggacctgtgacaggaatcttctctggttatgttaattccatgctgcaggaatacgcaaaaaatccatctgtcaactggaaacacaaagatgcagccatctacctagtgacatctttggcatcaaaagcccaaacacagaagcatggaattacacaagcaaatgaacttgtaaacctaactgagttctttgtgaatcacatcctccctgatttaaaatcagctaatgtgaatgaatttcctgtccttaaagctgacggtatcaaatatattatgatttttagaaatcaagtgccaaaagaacatcttttagtctcgattcctctcttgattaatcatcttcaagctgaaagtattgttgttcatacttacgcagctcatgctcttgaacggctctttactatgcgagggcctaacaatgccactctctttacagctgcagaaatcgcaccgtttgttgagattctgctaacaaaccttttcaaagctctcacacttcctggctcttcagaaaatgaatatattatgaaagctatcatgagaagtttttctctcctacaagaagccataatcccctacatccctactctcatcactcagcttacacagaagctattagctgttagtaagaacccaagcaaacctcactttaatcactacatgtttgaagcaatatgtttatccataagaataacttgcaaagctaaccctgctgctgttgtaaattttgaggaggctttgtttttggtgtttactgaaatcttacaaaatgatgtgcaagaatttattccatacgtctttcaagtgatgtctttgcttctggaaacacacaaaaatgacatcccgtcttcctatatggccttatttcctcatctccttcagccagtgctttgggaaagaacaggaaatattcctgctctagtgaggcttcttcaagcattcttagaacgcggttcaaacacaatagcaagtgctgcagctgacaaaattcctgggttactaggtgtctttcagaagctgattgcatccaaagcaaatgaccaccaaggtttttatcttctaaacagtataatagagcacatgcctcctgaatcagttgaccaatataggaaacaaatcttcattctgctattccagagacttcagaattccaaaacaaccaagtttatcaagagttttttagtctttattaatttgtattgcataaaatatggggcactagcactacaagaaatatttgatggtatacaaccaaaaatgtttggaatggttttggaaaaaattattattcctgaaattcagaaggtatctggaaatgtagagaaaaagatctgtgcggttggcataaccaaattactaacagaatgtcccccaatgatggacactgagtataccaaactgtggactccattattacagtctttgattggtctttttgagttacccgaagatgataccattcctgatgaggaacattttattgacatagaagatacaccaggatatcagactgccttctcacagttggcatttgctgggaaaaaagagcatgatcctgtaggtcaaatggtgaataaccccaaaattcacctggcacagtcacttcacaagttgtctaccgcctgtccaggaagggttccatcaatggtgagcaccagcctgaatgcagaagcgctccagtatctccaagggtaccttcaggcagccagtgtgacactgctttaaactgcatttttctaatgggctaaacccagatggtttcctaggaaatcacaggcttctgagcacagctgcattaaaacaaaggaagttctccttttgaacttgtcacgaattccatcttgtaaaggatattaaatgttgctttaacctgaaccttgagcaaattagttggtttgtgtgatcatacagttatgtgggtggcttctagtttgcaacttcaagggacaagtattaatagttcagtgtatggcgttggtttgtgttgagcgtttgcacggtttggataatcttaaattttgacggacactgtggagactttctgttactaaatccttttgttttgaagctgttgctatttgtatttctcttgtcctttatattttttgtctgtttatttacgcttttattggaaatgtgaataagtaaagaattacttgtgttacttgccaagcagtgcacatttcatagtttcaaatctgtaatcagcaataaaaatcctaaaatatgtacctaaaaaaaaaaaaaaaaaaaa

SEQ ID NO: 4 - DNA, 1290 bp, Homo sapiens
gcggcggcggcggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaa

SEQ ID NO: 5 - DNA, 3966 bp, Homo sapiens
gggcggcgccgccgcgcccgacgcctctgggggtggggacttcgcgggagtcgccggcggaggcgaggaggagctctgcgcggcgcggcgggcgatccgagccgggacgggctgcaggcgggggtgctgcagaggacacgaggcggcgggctggagacatggaccgcggcgagcaaggtctgctgagaacagacccagtccctgaggaaggagaagatgttgctgccacgatcagtgccacagagaccctctcggaagaggagcaggaagagctaagaagagaacttgcaaaggtagaagaagaaatccagactctgtctcaagtgttagcagcaaaagagaagcatctagcagagatcaagcggaaacttggaatcaattctctacaggaactaaaacagaacattgccaaagggtggcaagacgtgacagcaacatctgcttacaagaagacatctgaaaccttatcccaggctggacagaaggcctcagctgctttttcgtctgttggctcagtcatcaccaaaaagctggaagatgtaaaaaactccccaacttttaaatcatttgaagaaaaggtcgaaaacttaaagtctaaagtagggggaaccaagcctgctggtggtgattttggagaagtcttgaattcggctgcaaatgctagtgccaccaccacggagcctcttccagaaaagacacaggagagcctgtgagattcctacctttgttctgctacccactgccagatgctgcaagcgaggtccaagcacatcttgtcaacatgcattgccatgaatttctaccagatgtgcttttatttagctttacatattcctttgaccaaatagtttgtgggttaaacaaaatgaaaatatcttcacctctattcttgggaaacaccctttagtgtacatttatgttcctttatttaggaaacaccattataaaaacacttatagtaaatggggacattcactataatgatctaagaagctacagattgtcatagttgttttcctgctttacaaaattgctccagatctggaatgccagtttgacctttgtcttctataatatttcctttttttcccctctttgaatctctgtatatttgattcttaactaaaattgttctcttaaatattctgaatcctggtaattaaaagtttgggtgtattttctttacctccaaggaaagaactactagctacaaaaaatattttggaataagcattgttttggtataaggtacatattttggttgaagacaccagactgaagtaaacagctgtgcatccaatttattatagttttgtaagtaacaatatgtaatcaaacttctaggtgacttgagagtggaacctcctatatcattatttagcaccgtttgtgacagtaaccatttcagtgtattgtttattataccacttatatcaacttatttttcaccaggttaaaattttaatttctacaaaataacattctgaatcaagcacactgtatgttcagtaggttgaactatgaacactgtcatcaatgttcagttcaaaagcctgaaagtttagatctagaagctggtaaaaatgacaatatcaatcacattaggggaaccattgttgtcttcacttaatccatttagcactatttaaaataagcacaccaagttatatgactaatataacttgaaaattttttatactgaggggttggtgataactcttgaggatgtaatgcattaataaaaatcaactcatcattttctacttgttttcaatgtgttggaaactgtaaaatgatactgtagaacctgtctcctactttgaaaactgaatgtcagggctgagtgaatcaaagtgtctagacatatttgcatagaggccaaggtattctattctaataactgcttactcaacactaccaccttttccttatactgtatatgattatggcctacaatgttgtatttgttatttattaaattgtgattgttttattattgtttatgccaaatgttaactgccaagcttggagtgacctaaagcattttttaaaagcatggctagatttacttcagtataaattatcttatgaaaaccaaattttaaaagccacaggtgttgattgttataaaataacatgctgccattcttgattgctagagtttttgttagtactttggatgcaattaaaactatgtgctatcacatgtgaaaagcttaataaattccatctatcagtagtataggtctcaatatttattatgagaccagtggtctggaaacagcttgttgtaccgaatcaactggagtctatgcttaaaaaaaaaaaatttttttttaaccatccttaaattattgcttaatggtatcatattaacatattctaaataagggctttaaggcacaggctgttgaagcattttctcagaggagtggatctgtagaagtctgtctttctatagaaatattgtgcttactcaagtgttaaattattttttctatgaactagtctacttcttaaaattcaaacatattcttttgatcacattgtttcttgagcatcctgccctgctactaacttttcaacaaggcaaaatggagtaaagtggcaatttctttagatgagtgaaataccctcaagtctcttttctgcccaaaaagggaaaagtgatagaaatgggggtggcaagtggggtgagtggatgaaggtgggtattgggggtggctgtgaaagaaaataatggagaatcacttttctagacatctacctatacttaatctaagaaacaaagtaatctactgtaaagtactctgccccttgaaagaagtattaaaaagagtgaggatggatttagaaaaaaacatgaatttagaaatattcaaaatggtttttgtggcagattcaatattatgaattcacagatatttaaagaatgagaaacatagtaattagtagaaatgccagaaacagttcctggttcctcttgtgtttgacactaagaaaatagcaagagtgtgaaatctcagatacttatgaaatctcacagatgtaaggactcaagtgtagaagaaaatatccccttcttacaaaaagaaatgtcaatttatggagtttgtgggaaatagggcaagaattcttatgcttatgagagccaagtagtcagtggaagagagtagagctcaaaactggattatcaccttagcaacttagaatagtttgaaatagaaaaaaagtatttaatttggatctggatctgttaagatatgcacagtctattttttgtatagtattggaaaataaaaatgctataatttgggtatgggttcttttgtatacattacctgcccattgaggaaaggggcaagtccattatgcaacttctctccaaacccttcatattctggatatgatacttaaagagccatgagcagctacattctgcctatcagaaggcatttattaaatgactgcttgctgaagatgtgtccaaagagatcctacattaaggtcatttgtcagaatgatgtttgtgtttgtttagagttggctgacctccaactcctggggtcaaggaatcctactgccttagcttcccaaatagctaggactataggcatgcacccggccatgtgttttatttatagctcttaaagcccagatgaagaaatcacatttttgcccatagtgaagaaacatttggccattgattagtccttattttcagtgactgtcctgttttcattagattagagagaccctgtgtgggccacagttaatataaaccattatcacttttaagtaaacctgcacatcttagatttcataatttccttattgttctgactcaaaatgaactaagagcttttcactttttgtttgtaagttctcagagagtcgggtctgcaagtgcttttgcctgccaggattttgtactcaaataaatgctatttggcaactaaaaaaaaa

SEQ ID NO: 6 - DNA, 902 bp, Homo sapiens
cacccgttcgcccgtccccctgccgcattcacaatgcagcctgcttttgcaaagtggtacgatcgaagggactgtgtcttcactgaatcttgtgttgaagacaataaggatgttaatgtaaattttgaaaaatccaaacttacattcagttgtcttggaggaagtgataattttaagcatttaaatgaaattggtcttttttactctattgatccaaatgattccaagcataaaagaacggacagatcaattttatgttgtttacgaaaaggagaatctggccagtcatggccaaggttaacaaaagaaagggcaaagcttaactggcttagtatggacttcaatcattggaaagactgggaagatggttcagatgaagacaggtctaattttgatcgtttctccgagatgatgaacaacatgggtggggatgaggatgtagatttaccagaagtagatgaagcaggtgatgattcacaagacagtgatgatgaaaaaatgccagatctggagtaagaaatatttcatcacctggattttgagaaagaaaataacttctctgcaagatttcataattgagagaattcctcagttgatagccctaaaggcagatgctgtatttgcctactttaacccatttttcaacctgtttgttttttaaaaggcttcactaagggtttatatgtaccattgtatggagcaatttgaagtcagctaaggcaataaccttatgcatgaacatttcccagactttcatgaagctgttgaggtcctaggcaattgatgcagcagttgtgataaataaaaacatctcacctaagtctccttttcttcataacatagatactgacgtgataggaagctctgggcttaggtaaagagaacaaaattttagtttatag

SEQ ID NO: 7 - DNA, 2649 bp, Homo sapiens
aggaggacctgggggtgtggcagcgaggaagggccgagccacggactgtggggccgaaactcgctcccgcccaccctttctcgaggctgtggcctccgcgagagccgagcgggccgcaccgccggccgtgcgactgccccagtcagacacgaccccggcttctagcccgcctaagcctgtttggggttgctgactcgtttcctccccgagtttcccgcgggaactaactcttcaagaggaccaaccgcagcccagagcttcgcagacccggccaaccagaggcgaggttgagagcccggcgggccgcggggagagagcgtcccatctgtcctggaaagcctgggcgggtggattgggaccccgagagaagcaggggagctcggcggggtgcagaagtgcccaggcccctccccgctggggttgggagcttgggcaggccagcttcacccttcctaagtccgcttctggtctccgggcccagcctcggccaccatgtcccgccagaccacctctgtgggctccagctgcctggacctgtggagggaaaagaatgaccggctcgttcgacaggccaaggtggctcagaactccggtctgactctgaggcgacagcagttggctcaggatgcactggaagggctcagagggctcctccatagtctgcaagggctccctgcagctgttcctgttcttcccttggagctgactgtcacctgcaacttcattatcctgagggcaagcttggcccagggtttcacagaggatcaggcccaggatatccagcggagcctagagagagtgctggagacacaggagcagcaggggcccaggttggaacaggggctcagggagctgtgggactctgtccttcgtgcttcctgccttctgccggagctgctgtctgccctgcaccgcctggttggcctgcaggctgccctctggttgagtgctgaccgtcttggggacctggccttgttactagagaccctgaatggcagccagagtggagcctctaaggatctgctgttacttctgaaaacttggagtcccccagctgaggaattagatgctccattgaccctgcaggatgcccagggattgaaggatgtcctcctgacagcatttgcctaccgccaaggtctccaggagctgatcacagggaacccagacaaggcactaagcagccttcatgaagcggcctcaggcctgtgtccacggcctgtgttggtccaggtgtacacagcactggggtcctgtcaccgtaagatgggaaatccacagagagcactgttgtacttggttgcagccctgaaagagggatcagcctggggtcctccacttctggaggcctctaggctctatcagcaactgggggacacaacagcagagctggagagtctggagctgctagttgaggccttgaatgtcccatgcagttccaaagccccgcagtttctcattgaggtagaattactactgccaccacctgacctagcctcaccccttcattgtggcactcagagccagaccaagcacatactagcaagcaggtgcctacagacggggagggcaggagacgctgcagagcattacttggacctgctggccctgttgctggatagctcggagccaaggttctccccacccccctcccctccagggccctgtatgcctgaggtgtttttggaggcagcggtagcactgatccaggcaggcagagcccaagatgccttgactctatgtgaggagttgctcagccgcacatcatctctgctacccaagatgtcccggctgtgggaagatgccagaaaaggaaccaaggaactgccatactgcccactctgggtctctgccacccacctgcttcagggccaggcctgggttcaactgggtgcccaaaaagtggcaattagtgaatttagcaggtgcctcgagctgctcttccgggccacacctgaggaaaaagaacaaggggcagctttcaactgtgagcagggatgtaagtcagatgcggcactgcagcagcttcgggcagccgccctaattagtcgtggactggaatgggtagccagcggccaggataccaaagccttacaggacttcctcctcagtgtgcagatgtgcccaggtaatcgagacacttactttcacctgcttcagactctgaagaggctagatcggagggatgaggccactgcactctggtggaggctggaggcccaaactaaggggtcacatgaagatgctctgtggtctctccccctgtacctagaaagctatttgagctggatccgtccctctgatcgtgacgccttccttgaagaatttcggacatctctgccaaagtcttgtgacctgtagctgccacgttttgaagagcttgagctgggtccccagtgggctgtctctctgtggggagggctttctgcttcaccatcattaggaatgtgaccattcctatataattcctggactggtgagattggtggtaggcctgtgaaatttgccctagttactaccattctcgttttggaggaaacaatctctgccaccaccaagtcattgactttgctcgaggcaccttttttcctgtttctccttttctgttgtcgagtaaaatttcatatttataaaaaaaaaaaaaaaaaa

SEQ ID NO: 8 - DNA, 1709 bp, Homo sapiens
tcctcacaattgctctacagctcagagcagcaactgctgaggctgccttgggaagaagatgatcctaaacaaagctctgctgctgggggccctcgccctgactgccgtgatgagcccctgtggaggtgaagacattgtggctgaccatgttgcctcctatggtgtgaacttctaccagtctcacggtccctctggccagtacacccatgaatttgatggagacgaggagttctacgtggacctggagacgaaagagactgtctggcagttgcctatgtttagcaaatttataagttttgacccgcagagtgcactgagaaatatggctgtgggaaaacacaccttggaattcatgatgagacagtccaactctaccgctgccaccaatgaggttcctgaggtcacagtgttttccaagtttcctgtgacgctgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggacgagcctcttctgaaacactgggagcctgagattccagcccctatgtcagagctcacagagactttggtctgcgccctggggttgtctgtgggcctcatgggcattgtggtgggcactgtcttcatcatccaaggcctgcgttcagttggtgcttccagacaccaagggctcttatgaatctcatcctgaaaggaaggtgcatcaccatctacaggagaagaagaatggacttgctaaatgacctagcactattctctggcctgatttatcatatcccttttctcctccaaatgtttcttctctcacctcttctctgggacttaaggtgctatattccctcagagctcacaaatgcctttcaattctttccctgacctcctttcctgaatttttttattttctcaaatgttacctactaagggatgcctgagtaagccactcagctacctaattcctcaatgacctttatctaaaatctccatggaagcaataaattcccttttgatgcctctattgaatttttcccatctttcatctcagggctgactgagagcataacttagaatgggtgactcttatgttttaggccaatttcatgtcattccccagatcatatttcatgtccagtaacacaggagcaaccaagtacagtgtatcctgataatttgttgatttcttaactggtgttaatatttctttcttccttttgttcctacccttggccactgccacccacccctcaattcaggtaccaacgaaccctctgcccttggctcagaatggttatagcagaaatacaaaaaaaaaaaaaaaagtctgtactaatttcaatatggctcttaaaaggaatgacagagaaataggatacaagaattttgaatctcaaaagttatcaaaagtaaaaaattttgttaccaaaagtcaaactgcattctcaaaactttaaatttgtgaagaatgacaacagtagaagctttcctctccccttctcaccttgaggagataaaaattctctaggcaggaaaagaaatggaagccagttagaaaaacattgaaataa

SEQ ID NO: 9 - DNA, 484 bp, Homo sapiens
atgaaacacctgtggttcttccttctcctggtggcagctcccawgatgggtcctgtcccaggtgcagctgcaggagtcgggcccaggcctggtgaagccttcggagaccctgtccctcatctgcactgtctctggtggctccatcaattcttactactggagctggctccggcagtcccccgggaagggactggagtggattggatatgtctattacaggggctccaactacaatccctccctcaagagtcgagtcagcatatcagaagacacgaccaagaaccagttctccctgaggctgagctctgtgaccgctgcggacacggccgtctactactgttcgagagatcgtgaccctaggaactactggtacttcgatctctggggccgtggcgccctggtcactgtctcctcagcctccaccaagggcccatcggtcttccccctggcaccctcctccaagagcacctctgggggcacagcg

SEQ ID NO: 10 - DNA, 1942 bp, Homo sapiens
gcagacgctcgggggaacatggcggctgcggagccggcggtccttgcgctccccaacagcggcgccgggggcgcgggggcgccgtcgggcacagtcccggtgctcttctgtttctcagtcttcgcgcgaccctcgtcggtgccacacggggcgggctacgagctgctcatccagaagttcctcagcctgtacggcgaccagatcgacatgcaccgcaaattcgtggtgcagctgttcgccgaggagtggggccagtacgtggacttgcccaagggcttcgcggtgagcgagcgctgcaaggtgcgcctcgtgccgttgcagatccagctcactaccctgggaaatcttacaccttcaagcactgtgtttttctgctgtgatatgcaggaaaggttcagaccagccatcaagtattttggggatattattagcgtgggacagagattgttgcaaggggcccggattttaggaattcctgttattgtaacagaacaataccctaaaggtcttgggagcacggttcaagaaattgatttaacaggtgtaaaactggtacttccaaagaccaagttttcaatggtattaccagaagtagaagcggcattagcagagattcccggagtcaggagtgttgtattatttggagtagaaactcatgtgtgcatccaacaaactgccctggagctagttggccgaggagtcgaggttcacattgttgctgatgccacctcatcaagaagcatgatggacaggatgtttgccctcgagcgtctcgctcgaaccgggatcatagtgaccacgagtgaggctgttctgcttcagctggtagctgataaggaccatccaaaattcaaggaaattcagaatctaattaaggcgagtgctccagagtcgggtctgctttccaaagtataggacatttgaagaactggtatgctactcactggtgaaggacagtcaggtgaaggactgtaagcccacacaagctcttcttatctctactagaattaaaatgttaagtcaaaaacggctccttttttgcgcctcctagtgaaacttaaccagctagaccatttgagtaccagcatttagttacaaacgtcaaaggcttccggtgctgcttaccttccttttttgttaatgtgcttttatttattaaaaaaaattacaatgaagatgcctgttttgtctctactgtgtactctgatcgtatctttccaaagtgcagactcttgtgaagttttcttaaattgttcactttaaagaaaatgacgtaccaacaatgatttggcttttatattactgtaagatgttataatgttaatgtggatgtagtgcttttactttacagattgattggaataagattattgcatatgaatttacccacaggactctgaatcatgttacccactcccctcacaatgttgtccacttagtgagttgcattgatctatccgtaccaaatgatgttgaataattacatatctttcttgactatactgatttcttattttggtcactattactaaatctctgttaatattctctcttttaactgaaaagggatgggatagaagggtttgcaatgccatattattggtggagggctgttttaacatctttgaagtatggcttgctgaatatctttaccaacatcttgaatatatattctagtgtccacaagatttagcaaaaagataaagcttgggtggaatatcattttaaaatgttcatgttctgttctatattttcttcacctactctccaaatattgtaatgcaaaaagtctcagtaatgatttggtagtattaattttgtggtcattgtttctcttcgataaatttattttcattaaatacttgttagagggttttgaaatgtttttcaaatatgtgaaatgtgaaactgctgtcttttatattaaagtaattaaagaaaa

SEQ ID NO: 11 - DNA, 1147 bp, Homo sapiens
cggcggtcggtcagcgcacagggcacggcttcctctgcttctcccaccacttagtctcaacccgggtgtgtttgcactactagagaatgaaatatgactgaggactctcagagaaactttcgttcagtatattatgagaaagtggggtttcgtggagttgaagaaaagaaatcattagaaattctcctaaaagatgaccgtctggatactgagaaactttgtacttttagtcagaggttccctctcccgtccatgtaccgtgcattggtatggaaggtgcttctaggaatcttgcctccacaccacgagtcccatgccaaggtgatgatgtatcgtaaggagcagtacttggatgtccttcatgccctgaaagtcgttcgctttgttagtgatgccacacctcaggctgaagtctatctccgcatgtatcagctggagtctgggaagttacctcgaagtccctcttttccactggagccagatgatgaagtgtttcttgccatagctaaagccatggaggaaatggtggaagatagtgtcgactgttactggatcacccgacgctttgtgaaccaattaaataccaagtaccgggattccttgccccagttgccaaaagcgtttgaacaatacttgaatctggaagatggcagactgctgactcatctgaggatgtgttccgcggcgcccaaacttccttatgatctctggttcaagaggtgctttgcgggatgtttgcctgaatccagtttacagagggtttgggataaagttgtgagtggatcctgtaagatcctagtttttgtagctgtcgaaattttattaacctttaaaataaaagttatggcactgaacagtgcagagaagataacaaagtttctggaaaatattccccaggacagctcagacgcgatcgtgagcaaggccattgacttgtggcacaaacactgtgggaccccggtccattcaagctgaacgcacccgctggttgtggaccgtctgccaggcaccacagtgagcattgtgttcttggcatgtgatctgggaaactgattgaataatacacttttcttgctttggtgctcaaagtggtttttttcccccaataaaattatttaattgaaaaaaaaaaaaaaaaaaaaaaaaa

SEQ ID NO: 12 - DNA, 3089 bp, Homo sapiens
gtagggctagagttctggccgtggcgggccggtttctgcgtgctgcgtgcgcggccgcgtgggctatggggcggcggtcgcggggtcggcggctccagcaacagcagcggccggaggacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtgagcaggcccaaggcagcgggggcccacacccctcacacgcaaaactggcttcttctggtcactggtgtctgaaaccaaatccagagcagcctgtggcctgtaaagcatatatttctaatgactgcagactggtgggatcataggagccttctgaatgaccaggactgctttctttggagctgatgaaaatgtactcttttagcgtgttagaaatcacttgttttattttgtttctttggccaagctgggtctagtgtttcttttgctgggaatagactttcaaaagttgtacttctatcaagaaacaaaactgcccttgcagaaatttcaggtcttttgttaagcctgtattggtcttaaggtgcagtattttttaaattattatttatagaaagaatctataaattcttggggaagtgtgttataagctttaataattacattgagctgcacctcagtggtgtgtcattaacatgcagtggggttaatatctgaggcctcagatgactttgtgccttttggaataaagggtaaaataaactctcccagagtaagagctgtatcgtgaattgtcatactaattattgagggggacttatgtgcttttattgaatggagtgctttacaatttttatttttaaatggggttgggatccttggaatatttcaataaaattgataaaatataaaaaaaaaaaaaaaaaaaaaaaaaaaaaaaaaa

SEQ ID NO: 13 - DNA, 2077 bp, Homo sapiens
agcggagtctgtgattggctgaagctcagctacttgtcacaagaatatactctcagttgcagttaatttacatactaagttaggttgcagttccctacatagcaactcagagtagggaggaaattttaggccaaatttaatttaaattatgttggagcatgaccaaagttggtcatggccagaagctgaacacagatatggaagctaacacaatatccagtggagagagattgtggtagagtacatggggcgaaatggttgatttctgcatttttgttggtgacgccatcaggatttgctgaagaaccaaacgtgtgacaggaaaggacaacacaaggagctgccagctttggaaaccagttctactctcagtattaccttcgcaaaagatagagctttgtaaatttctagattcctcgttcttattatcctagactatttaaccagaatctccagggtcgtggtcctggaaacaatacgtttaaaactcttcccaaggatccttcagcagccaacttgactttggctaaggagtccgccagctctgggctcacttgggttgtttgcaaggaagagcccatgcccagagtataaattgggagaacagcgctgtgcaaagagcgtgggttggtgagctagtggatggcgagattaatctcactctgatccctaaagcagggtgcagtaatactctaaatgcctcaaatgggttgggaaccatggaaggaatcccggaggagcccgatcttaacgaagcttttctcaggcctttggtaggaggacagaaacgaagttgaatgaaaaagacagtggagtgggactgagaaatgacaacggcccaccacagacctctctctttcaaggcctgtgtcgtcaggacgcaggccagaagaagccacgacgaggcgcaggcgcgactccacgagggccacgcccctcctagagaaggaccgacccgttttccgcccgcagcggagcttgggtttccgggaggacctgattgtgcaagggaccaaaccggatagaggtcgcgcgccctctttcgtctgccttccggttcactaatacgcaagttcgcagaagtgcgggaacgcgccgtccctctgcgcaggcgcagtcggcggtcggcgtggggcgctatgccggggcggcacgtttctcgagtccgggcattgtacaagcgcgtcttgcagctgcaccgtgttctgcccccggacctcaaatccctgggcgaccagtacgtgaaagacgaatttaggagacataagaccgttggttctgacgaggcacagcgtttcttgcaagaatgggaggtgtatgcaacagcgttattgcaacaggctaacgaaaacagacaaaattcaactggaaaagcatgttttggcaccttcctcccagaagaaaaacttaatgactttcgtgatgaacaaattggacagttgcaggagctgatgcaagaagccacaaaacccaataggcaatttagtatttctgagtctatgaaaccaaaattttagtctatacaacaaagcttaataagacatgcaaaaatttagaacccctactttaactgtcattggtttttgaaatatatttaagctttgaaaacacctgttattaatgaaatactcttttattttggatattatgattgcagtatatggatcaagatcactagtgacaattgaaaaaaactattggaataatagcacttgtatgaaattcagttttggaactaaacagcaaatttctagaattttgctgaaaatgttttaaaatgctattctcatccagccatattagtcttctggcttttctttagcttcatcaaataagcatgttgtgataatgatagatgtacaattccaacaaggttattattttttaaatacattgtcattctgaacattttatcacttctagtttaataatacatacatgatttttcttctgaatgtctcttctccctgcatcactgttcattcacaatgaaaggttaggaagaagctttaaaattcactattttactatcaatcatttgtataataaactatacaaagtaaaaaaaaaaaaaaaa

SEQ ID NO: 14 - DNA, 1042 bp, Homo sapiens
cgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtggggcaggggtttcgggctggtggagcatgtgctgggacaggacagcatcctcaatcaatccaacagcatattcggttgcatcttctacacactacagctattgttaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcaaaaaaaaaaaaaaaaaaaaaaaaaaaaaaaaaaaaaaaaaaaaa

SEQ ID NO: 15 - DNA, 2970 bp, Homo sapiens
gccgcggcgagagcgcgcccagccccgccgcgatgcccgcgcgcccaggacgcctcctcccgctgctggcccggccggcggccctgactgcgctgctgctgctgctgctgggccatggcggcggcgggcgctggggcgcccgggcccaggaggcggcggcggcggcggcggacgggccccccgcggcagacggcgaggacggacaggacccgcacagcaagcacctgtacacggccgacatgttcacgcacgggatccagagcgccgcgcacttcgtcatgttcttcgcgccctggtgtggacactgccagcggctgcagccgacttggaatgacctgggagacaaatacaacagcatggaagatgccaaagtctatgtggctaaagtggactgcacggcccactccgacgtgtgctccgcccagggggtgcgaggataccccaccttaaagcttttcaagccaggccaagaagctgtgaagtaccagggtcctcgggacttccagacactggaaaactggatgctgcagacactgaacgaggagccagtgacaccagagccggaagtggaaccgcccagtgcccccgagctcaagcaagggctgtatgagctctcagcaagcaactttgagctgcacgttgcacaaggcgaccactttatcaagttcttcgctccgtggtgtggtcactgcaaagccctggctccaacctgggagcagctggctctgggccttgaacattccgaaactgtcaagattggcaaggttgattgtacacagcactatgaactctgctccggaaaccaggttcgtggctatcccactcttctctggttccgagatgggaaaaaggtggatcagtacaagggaaagcgggatttggagtcactgagggagtacgtggagtcgcagctgcagcgcacagagactggagcgacggagaccgtcacgccctcagaggccccggtgctggcagctgagcccgaggctgacaagggcactgtgttggcactcactgaaaataacttcgatgacaccattgcagaaggaataaccttcatcaagttttatgctccatggtgtggtcattgtaagactctggctcctacttgggaggaactctctaaaaaggaattccctggtctggcgggggtcaagatcgccgaagtagactgcactgctgaacggaatatctgcagcaagtattcggtacgaggctaccccacgttattgcttttccgaggagggaagaaagtcagtgagcacagtggaggcagagaccttgactcgttacaccgctttgtcctgagccaagcgaaagacgaactttaggaacacagttggaggtcacctctcctgcccagctcccgcaccctgcgtttaggagttcagtcccacagaggccactgggttcccagtggtggctgttcagaaagcagaacatactaagcgtgaggtatcttctttgtgtgtgtgttttccaagccaacacactctacagattctttattaagttaagtttctctaagtaaatgtgtaactcatggtcactgtgtaaacattttcagtggcgatatatcccctttgaccttctcttgatgaaatttacatggtttcctttgagactaaaatagcgttgagggaaatgaaattgctggactatttgtggctcctgagttgagtgattttggtgaaagaaagcacatccaaagcatagtttacctgcccacgagttctggaaaggtggccttgtggcagtattgacgttcctctgatcttaaggtcacagttgactcaatactgtgttggtccgtagcatggagcagattgaaatgcaaaaacccacacctctggaagataccttcacggccgctgctggagcttctgttgctgtgaatacttctctcagtgtgagaggttagccgtgatgaaagcagcgttacttctgaccgtgcctgagtaagagaatgctgatgccataactttatgtgtcgatacttgtcaaatcagttactgttcaggggatccttctgtttctcacggggtgaaacatgtctttagttcctcatgttaacacgaagccagagcccacatgaactgttggatgtcttccttagaaagggtaggcatggaaaattccacgaggctcattctcagtatctcattaactcattgaaagattccagttgtatttgtcacctggggtgacaagaccagacaggctttcccaggcctgggtatccagggaggctctgcagccctgctgaagggccctaactagagttctagagtttctgattctgtttctcagtagtccttttagaggcttgctatacttggtctgcttcaaggaggtcgaccttctaatgtatgaagaatgggatgcatttgatctcaagaccaaagacagatgtcagtgggctgctctggccctggtgtgcacggctgtggcagctgttgatgccagtgtcctctaactcatgctgtccttgtgattaaacacctctatctcccttgggaataagcacatacaggcttaagctctaagatagataggtgtttgtccttttaccatcgagctacttcccataataaccactttgcatccaacactcttcacccacctcccatacgcaaggggatgtggatacttggcccaaagtaactggtggtaggaatcttagaaacaagaccacttatactgtctgtctgaggcagaagataacagcagcatctcgaccagcctctgccttaaaggaaatctttattaatcacgtatggttcacagataattctttttttaaaaaaacccaacctcctagagaagcacaactgtcaagagtcttgtacacacaacttcagctttgcatcacgagtcttgtattccaagaaaatcaaagtggtacaatttgtttgtttacactatgatactttctaaataaactctttttttttaaaaaaaaaaaaaaaa

SEQ ID NO: 16 - DNA, 2323 bp, Homo sapiens
gcgagctcttgcagcctccccgcccctcccgcaacgctcgaccccaggattcccccggctcgcctgcccgccatggccgacaaggaagcagccttcgacgacgcagtggaagaacgagtgatcaacgaggaatacaaaatatggaaaaagaacaccccttttctttatgatttggtgatgacccatgctctggagtggcccagcctaactgcccagtggcttccagatgtaaccagaccagaagggaaagatttcagcattcatcgacttgtcctggggacacacacatcggatgaacaaaaccatcttgttatagccagtgtgcagctccctaatgatgatgctcagtttgatgcgtcacactacgacagtgagaaaggagaatttggaggttttggttcagttagtggaaaaattgaaatagaaatcaagatcaaccatgaaggagaagtaaacagggcccgttatatgccccagaacccttgtatcatcgcaacaaagactccttccagtgatgttcttgtctttgactatacaaaacatccttctaaaccagatccttctggagagtgcaacccagacttgcgtctccgtggacatcagaaggaaggctatgggctttcttggaacccaaatctcagtgggcacttacttagtgcttcagatgaccataccatctgcctgtgggacatcagtgccgttccaaaggagggaaaagtggtagatgcgaagaccatctttacagggcatacggcagtagtagaagatgtttcctggcatctactccatgagtctctgtttgggtcagttgctgatgatcagaaacttatgatttgggatactcgttcaaacaatacttccaaaccaagccactcagttgatgctcacactgctgaagtgaactgcctttctttcaatccttatagtgagttcattcttgccacaggatcagctgacaagactgttgccttgtgggatctgagaaatctgaaacttaagttgcattcctttgagtcacataaggatgaaatattccaggttcagtggtcacctcacaatgagactattttagcttccagtggtactgatcgcagactgaatgtctgggatttaagtaaaattggagaggaacaatccccagaagatgcagaagacgggccaccagagttgttgtttattcatggtggtcatactgccaagatatctgatttctcctggaatcccaatgaaccttgggtgatttgttctgtatcagaagacaatatcatgcaagtgtggcaaatggcagagaacatttataatgatgaagaccctgaaggaagcgtggatccagaaggacaagggtcctagatatgtctttacttgttgtgattttagactccccttttttcttctcaaccctgagagtgatttaacactggttttgagacagactttattcagctatccctctatataataggtaccaccgataatgctattagcccaaaccgtgggtgttttctaaatattaataggggggcttgattcaacaaagccacagacttaacgttgaaattttcttcaggaattttctagtaacccaggtctaaagtagctacagaaaggggaatattatgtgtgattatttttcttcttatgctatatccccaagtttttcagactcatttaagtaaaggctagagtgagtaaggaatagagccaaatgaggtaggtgtctgagccatgaagtataaatactgaaagatgtcacttttattcaggaaatagggggagattcaagtcgtatagattcctactcgaaaatcttgacacctgactttccaggatgcacattttcatacgtagaccagtttcctcttggtttcttcagttaagtcaaaacaacacgttcctctttccccatatattcatatatttttgctcgttagtgtatttcttgagctgttttcatgttgtttatttcctgtctgtgaaatggtgtttttttttttgttgttggtttttttttttttttttaacttgggaccaccaagttgtaaagatgtatgtttttacctgacagttataccacaggtagactgtcaagttgagaagagtgaatcaataacttgtatttgttttaaaaattaaattaatccttgataagagttgcttttttttttaggagttagtccttgaccactagtttgatgccatctccattttgggtgacctgtttcaccagcaggcctgttactctccatgactaactgtgtaagtgcttaaaatggaataaattgcttttctacataaaaaaaaaaaaaaaaaaaaaaaaaaaaaa

SEQ ID NO: 17 - DNA, 2018 bp, Homo sapiens
aggcggctgtatctggagcagtcggggcgggcaggcccagctgagaggtgcgcgggcgaggacagcggcagcgatgcgggaatgcatatcagtccacgtgggccaagcgggagttcagattggcaatgcctgctgggagctcttctgcctggaacacggcatccaggcagacggcacttttgatgctcaagctagcaagatcaacgatgatgactccttcaccacctttttcagcgagactggcaatgggaagcatgtgccccgggccgtcatgatagatctggagcctactgtagtggatgaggttcgggcaggaacctaccgccagctcttccatccagagcagctgatcacaggaaaggaggatgcagccaacaactatgcccggggccactacacggtgggcaaggagagcattgacctggtgctggaccgcatacggaagctgacagatgcttgctctggcctgcagggcttcctgattttccacagttttggtgggggcactggctccggcttcacttctctgctgatggaacgcctctccctggattatggcaagaaatccaagctggagtttgccatctacccagccccccaggtctctactgcagtggtggagccctacaactccatcctgaccacccacaccacactggaacattcagattgtgctttcatggtggacaacgaagccatctatgacatctgccgcaggaaccttgacattgagcgccctacctataccaacctcaaccgcctcatcagtcagattgtgtcctcaatcactgcttctctccgctttgacggggccctcaatgtggacctcactgagttccagaccaacctggtgccctacccccgcatccacttcccgctggtcacctacgcgcccatcatctctgccgagaaagcctatcacgaacagctctctgtggccgagataaccagctcctgctttgagcccaacagccagatggtgaagtgcgacccgagacatggcaagtacatggcctgctgcatgctctaccggggcgacgtggtgcccaaggatgtgaatgtcgctattgctgccatcaagaccaagaggaccatccagtttgtagactggtgtcccacaggcttcaaggtgggcatcaactaccagcccccgaccgtggtccccgggggagacctggccaaggtgcagcgggccgtctgcatgctcagcaacaccacggccattgcggaggcctgggcccgcctcgaccacaagttcgacctcatgtacgccaagcgggcctttgtgcattggtatgtgggagaggggatggaagaaggagaattttctgaggccagggaagacttagctgccctggagaaggattatgaagaagtggggactgattcgtttgaagaagaaaatgaaggggaggaattttaaatatataccttccccttggctgtgtctctttatttatgctgtgccattcaaagcacatgttcaagagaacagaacactctccccgccccagcctgattcctgccttacccaggaggagggtgcctggccccagtacccagggtggcacgactgggctaagtggacactgagcttcatcaggccctccctgggtaggagcagctttgtgtcactaaagaaagtgagggccactgtctccggcgggtgaggctgcagcccagtttcacatgcgaggaggcctaatcaggagtttcaattccagatcgggctgggtccagccccaaaacatggcctgctggctggggagtgggaacactcagagaaaggggagatctgggcctgggaagattccggggcaggggtgagcggacgttcacatgagtgggtctatagcccgctccgggcatcatctagacttagcatgcattcactcccccatcacatattccaatacacaccctgccctaggcactgagagctggagagttggtgataagcgagacgaacacatttcctgccctcatacacacataaataaagtgaatgagatcatgtc

SEQ ID NO: 18 - DNA, 1704 bp, Homo sapiens
gcatttgactgcaactcttgtcgtcttatgtgggtgttgaattgatctgtctctgcaggccagatccaggctcctggaagaaccatgtccggcagctactggtcatgccaggcacacactgctgcccaagaggagctgctgtttgaattatctgtgaatgttgggaagaggaatgccagagctgccggctgaaaattacccaaccaagagaaatctgcaggatggactttctggtcctcttcttgttctacctggcttcggtgctgatgggtcttgttcttatctgcgtctgctcgaaaacccatagcttgaaaggcctggcaaggggaggagcacagatattttcctgtataattccagaatgtcttcagagagccgtgcatggattgcttcattaccttttccatacgagaaaccacaccttcattgtcctgcacctggtcttgcaagggatggtttatactgagtacacctgggaagtatttggctactgtcaggagctggagttgtccttgcattaccttcttctgccctatctgctgctaggtgtaaacctgttttttttcaccctgacttgtggaaccaatcctggcattataacaaaagcaaatgaattattatttcttcatgtttatgaatttgatgaagtgatgtttccaaagaacgtgaggtgctctacttgtgatttaaggaaaccagctcgatccaagcactgcagtgtgtgtaactggtgtgtgcaccgtttcgaccatcactgtgtttgggtgaacaactgcatcggggcctggaacatcaggtacttcctcatctacgtcttgaccttgacggcctcggctgccaccgtcgccattgtgagcaccacttttctggtccacttggtggtgatgtcagatttataccaggagacttacatcgatgaccttggacacctccatgttatggacacggtctttcttattcagtacctgttcctgacttttccacggattgtcttcatgctgggctttgtcgtggttctgagcttcctcctgggtggctacctgttgtttgtcctgtatctggcggccaccaaccagactactaacgagtggtacagaggtgactgggcctggtgccagcgttgtccccttgtggcctggcctccgtcagcagagccccaagtccaccggaacattcactcccatgggcttcggagcaaccttcaagagatctttctacctgcctttccatgtcatgagaggaagaaacaagaatgacaagtgtatgactgcctttgagctgtagttcccgtttatttacacatgtggatcctcgttttccaagcaaaaaaaaatggcttgtttgttttgatttctgctgtgcttataaatcactttcggtgggcaagggagagaggggaaaatgggtgttgactgaggaatcccccttgcttgtcttcttttgaaaccgggcatctctgaagtcctggtgtcaaggggatcaagagatgacttctcagaggttctaggtgatgctgagaccttggtgtctctaaactctgggcatgtggacaggaggggcttgcggccgtgtctctgacctgtgtgatgtgcaggagggtctcattgactcagcgcctgcgcgttagtgcctggctgtgctctcttttatgccccctctattctcctctctcccccaggggattttcatctcaacaacagagtg

SEQ ID NO: 19 - DNA, 4888 bp, Homo sapiens
gagagcggggacgtcagcgctgccagcgtggaaggagctgcggggcgcgggaggaggaagtagagcccgggaccgccaggccaccaccggccgcctcagccatggacgcgtccctggagaagatagcagaccccacgttagctgaaatgggaaaaaacttgaaggaggcagtgaagatgctggaggacagtcagagaagaacagaagaggaaaatggaaagaagctcatatccggagatattccaggcccactccagggcagtgggcaagatatggtgagcatcctccagttagttcagaatctcatgcatggagatgaagatgaggagccccagagccccagaatccaaaatattggagaacaaggtcatatggctttgttgggacatagtctgggagcttatatttcaactctggacaaagagaagctgagaaaacttacaactaggatactttcagataccaccttatggctatgcagaattttcagatatgaaaatgggtgtgcttatttccacgaagaggaaagagaaggacttgcaaagatatgtaggcttgccattcattctcgatatgaagacttcgtagtggatggcttcaatgtgttatataacaagaagcctgtcatatatcttagtgctgctgctagacctggcctgggccaatacctttgtaatcagctcggcttgcccttcccctgcttgtgccgtgtaccctgtaacactgtgtttggatcccagcatcagatggatgttgccttcctggagaaactgattaaagatgatatagagcgaggaagactgcccctgttgcttgtcgcaaatgcaggaacggcagcagtaggacacacagacaagattgggagattgaaagaactctgtgagcagtatggcatatggcttcatgtggagggtgtgaatctggcaacattggctctgggttatgtctcctcatcagtgctggctgcagccaaatgtgatagcatgacgatgactcctggcccgtggctgggtttgccagctgttcctgcggtgacactgtataaacacgatgaccctgccttgactttagttgctggtcttacatcaaataagcccacagacaaactccgtgccctgcctctgtggttatctttacaatacttgggacttgatgggtttgtggagaggatcaagcatgcctgtcaactgagtcaacggttgcaggaaagtttgaagaaagtgaattacatcaaaatcttggtggaagatgagctcagctccccagtggtggtgttcagatttttccaggaattaccaggctcagatccggtgtttaaagccgtcccagtgcccaacatgacaccttcaggagtcggccgggagaggcactcgtgtgacgcgctgaatcgctggctgggagaacagctgaagcagctggtgcctgcaagcggcctcacagtcatggatctggaagctgagggcacgtgtttgcggttcagccctttgatgaccgcagcagttttaggaactcggggagaggatgtggatcagctcgtagcctgcatagaaagcaaactgccagtgctgtgctgtacgctccagttgcgtgaagagttcaagcaggaagtggaagcaacagcaggtctcctatatgttgatgaccctaactggtctggaataggggttgtcaggtaaagtcttggcctgccgcttgatgttggagacttttctgtataaagaacatgagtgggtcattttctgaaccaccttagaaatccaagatgaacgtgtagtcacaatatgcttaacgaaaatgcaactcggttttctgggcatttacaaaagcacagtgcaagcaggcaatttggccagcgtggcgctcagggaggggagttgctgaatgttctgtctgtaggtgccgactctcagctacctaggagaagatgccaacaggtatatatcactcactgagaatggttactatccagggcatatagagaattgcaaatcagtaagaaaaagaacccagctctcggggagagaactgggtgcagtcctatggctgctttctcgccttcagaggtgaggcaaacctctttttgtattgcccatgagacttccagaccttgtatggaactaaagacttaggctttcagctgggcacggtggctcatgcctgtaatcccagcactttgggaggccaacaggagcatagatgacttgaggccaggagcttgagtccagcctggacaacatggcgaaaccccatctctagaaaaaatacaaaaattagccagaggtggtggtgcacacctgtagtcccagctactcgggaagcttagatggaaggatcaactgagcccaggaggtggaggttgcagtgagccaagatcatgccactgcaccccagcctgggccaccaagtgagaccctgctttaaaaaaaaaaaaaaaaggctttcctgtgactttctctttactcctggcacacttccagaaagtttgtggtctggagacactcgcagtagctcttttgcccactggttccactgcattgtcttgctaatatttagaggtttctaatcctgtatcagaagagacatttgatcttttaagctgaaatgctgtggtttgatgttgttttaggtatgaacatgctaatgatgataagagcagtttgaaatcagatcccgaaggggaaaacatccatgctggactcctgaagaagttaaatgaactggaatctgacctaacctttaaaataggccctgagtataagagcatgaagagctgcctttatgtcggcatggcgagcgacaacgtcgatgctgctgagctcgtggagaccattgcggccacagcccgggagatagaggagaactcgaggcttctggaaaacatgacagaagtggttcggaaaggcattcaggaagctcaagtggagctgcagaaggcaagtgaagaacggcttctggaagagggggtgttgcggcagatccctgtagtgggctccgtgctgaattggttttctccggtccaggctttacagaagggaagaacttttaacttgacagcaggctctctggagtccacagaacccatatatgtctacaaagcacaaggtgcaggagtcacgctgcctccaacgccctcgggcagtcgcaccaagcagaggcttccaggccagaagccttttaaaaggtccctgcgaggttcagatgctttgagtgagaccagctcagtcagtcacattgaagacttagaaaaggtggagcgcctatccagtgggccggagcagatcaccctcgaggccagcagcactgagggacacccaggggctcccagccctcagcacaccgaccagaccgaggccttccagaaaggggtcccacacccagaagatgaccactcacaggtagaaggaccggagagcttaagatgagactcattgtgtggtttgagactgtactgagtattgtttcagggaagatgaagttctattggaaatgtgaactgtgccacatactaatataaattactgttgtttgtgcttcactgggattttggcacaaatatgtgcctgaaaggtaggctttctaggaggggagtcagcttgtctaacttcatgtacatgtagaaccacgtttgctgtcctactacgacttttccctaagttaccataaacacattttattcacaaaaaacacttcgaatttcaagtgtctaccagtagcacccttgctctttctaaacataagcctaagtatatgaggttgcccgtggcaactttttggtaaaacagcttttcattagcactctccaggttctctgcaacacttcacagaggcgagactggctgtatcctttgctgtcggtctttagtacgatcaagttgcaatatacagtgggactgctagacttgaaggagagcagtgattgtgggattgtaaataagagcatcagaagccctccccagctactgctcttcgtggagacttagtaaggactgtgtctacttgagctgtggcaaggctgctgtctgggactgtcctctgccacaaggccatttctcccattatataccgtttgtaaagagaaactgtaaagtctcctcctgaccatatatttttaaatactggcaaagcttttaaaattggcacacaagtacagactgtgctcatttctgtttagtatctgaaaacctgatagatgctacccttaagagcttgctcttccgtgtgctacgtagcaccaacctggttaaaatctgaaaacaagtacccctttgacctgtctcccactgaagcttctactgccctggcagctcgcctgggcccaactcagaaacaggagccagcagagcactctctcacgctgatccagccgggcaccctgcttaagtcagtagaagctcgctggcactgcccgttcctacttttccgaagtactgcgtcactttgtcgtaagtaatggcccctgtgccttcttaatccagcagtcaagcttttgggagacctgaaaatgggaaaattcacactgggtttctggactgtagtattggaagccttagttatagtatattaagcctataattatactctgatttgatgggatttttgacatttacacttgtcaaaatgcagggggttttttttggtgcagatgattaaacagtcttccctatttggtgcaatgaagtatagcagataaaatgggggaggggtaaattatcaccttcaagaaaattacatgtttttatatatatttggaattgttaaattggttttgctgaaacatttcacccttgagatattatttgaatgttggtttcaataaaggttcttgaaattgttaaaaaaaaaaaaaaaaaaaaaaaaaaaaaaaaaaaagaaaaaaaaaaaaaaaaaa

SEQ ID NO: 20 - DNA, 2276 bp, Homo sapiens
ctcattgaactcgcctgcagctcttgggttttttgtggcttccttcgttattggagccaggcctacaccccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccataaaaaaaaaaaaaaaa

SEQ ID NO: 21 - DNA, 12227 bp, Homo sapiens
accccgggcaggtggagcgctcgcggctgctcgcggcgagcccgggagtgatggcgaggcccctgcgggcggccagcgcctgaggcgccccgccccgccccgccccgcgcggccctccccgcccggccctgccctgccctgctccctgccggcggctgcgggcgcttcctagtccgctcgggcggccgcccaggcgaggtgcggcctccgcacaggcggggggcgtaggcgcgcggggcccgccatggctgcgtcggagctctacacaaagtttgccagggtttggatacctgatccagaggaagtctggaagtcagcagagctgctcaaagattataagccaggagataaagtcctcctgcttcacctcgaggaaggaaaggatttggaataccatctagatccaaagaccaaggagctgcctcacttacgaaatcctgacatacttgttggtgaaaatgacctcacagccctcagctatcttcatgagcctgctgtgctccataatctcagagtccgctttattgattccaaacttatttatacgtattgtggtatagtcctagtagctataaatccctatgaacagctgcctatttatggagaagatattattaatgcatacagtggtcagaacatgggtgatatggatccacatatctttgcagtagctgaagaagcttacaagcaaatggccagagatgaacgaaatcagtccatcatcgtaagtggagagtctggggcaggaaaaacagtctcagctaagtatgccatgcgatactttgcaactgtgagtggttctgccagtgaggccaatgtggaggaaaaggtcttggcctccaaccccatcatggagtccattggaaatgctaaaacaaccaggaatgataatagcagccgttttgggaagtatattgagattggttttgataagagatatcgaatcattggtgccaatatgagaacttatcttttagagaaatccagagtggtattccaggcagaagaggagagaaactatcatatcttctatcagctttgtgcctcagcaaagttacctgaatttaaaatgctacgattaggaaatgcagataactttaattacacaaaacaaggaggcagtcctgtgattgaaggagtggatgatgcaaaggagatggcacatactaggcaggcctgcactttgctaggaattagtgaatctcatcaaatgggaattttccgaatacttgctggcatccttcacttaggcaatgttggatttacatcccgagatgcagacagctgcacaatacctcccaagcatgaacctctctgcatcttctgtgaactcatgggtgtggactatgaggagatgtgtcactggctctgccatcggaaactggctactgccacagagacatacatcaagcccatctccaagctgcaggccacaaatgcccgcgatgctttggccaagcacatctatgccaagctctttaactggattgtagataatgtcaatcaggctctccattctgctgtcaaacagcactcttttattggtgtgctagacatttacggatttgaaacatttgagataaatagttttgaacagttttgcataaattatgcaaatgaaaaactacagcaacaattcaatatgcatgtcttcaaattggagcaagaagaatatatgaaggaacaaattccatggacactcatagatttttatgataatcagccttgtattaatcttatagaatcaaaactaggcattctagatttactggatgaggaatgcaagatgcctaaaggcacagatgacacctgggcccaaaaattgtacaacacacatttgaacaaatgtgcactctttgaaaagcctcgtctatcaaacaaagctttcatcatccaacattttgctgacaaagtggaataccagtgtgaaggatttctcgaaaagaataaagacaccgtttttgaagaacaaattaaagttcttaaatcaagcaagtttaagatgctaccagaactatttcaagatgatgagaaggccatcagtccaacttcagccacctcctcagggcgcacacccctcacacgaactcctgcaaagcccaccaaaggcagaccaggccaaatggccaaagagcacaagaaaacagtggggcatcagttcagaaactccctgcacctgcttatggagacactcaatgccactacccctcactatgtgcgctgtatcaagcctaatgacttcaagttcccattcacgtttgatgagaagagggcagtgcagcagctgagagcatgtggtgtcctggaaaccatccgaatcagtgcggccggtttcccctcacggtggacttaccaagaatttttcagccgctaccgtgtcctaatgaagcagaaagatgtgctgagtgacagaaagcaaacatgcaagaatgtgttagagaaactgatactggacaaggacaaataccagtttggtaagacaaagatctttttccgtgccggtcaagtggcctatctagaaaaattgagagctgacaaactgagagctgcctgcatccggatccagaagaccatccgagggtggctgctgagaaagaagtacctacgcatgcggaaggcagccatcaccatgcagagatacgtgcggggctaccaggcccgatgctatgctaagtttctgcgcagaaccaaggcagcaaccatcattcaaaagtactggcgcatgtatgtggtccgcaggaggtacaagattagacgagctgccactatcgttcttcagtcttacttgcgaggcttcttggccagaaataggtatcgcaagatactccgtgagcacaaagcagtcatcattcagaagcgagtccggggctggctggcccgcacacactacaagaggagcatgcatgccatcatctaccttcagtgctgcttcaggcggatgatggccaagcgtgagctaaagaagctcaaaatcgaggctcgctcagtggagcgctataagaagctgcacatcggcatggagaacaagatcatgcagctgcagcgcaaagttgatgagcagaacaaagactacaaatgccttgtggagaaactaaccaatctggaaggaatatacaactctgagactgagaaactacgaagtgacttagaacgtcttcaactaagtgaagaggaagcgaaagttgccactgggcgggtccttagtctgcaggaagaaattgccaagctccggaaagacctggagcaaactcgttcagagaaaaaatgcattgaggaacatgcagatcgatacaaacaagaaacagagcagctggtatcaaatctgaaggaagaaaatactttgctgaagcaagaaaaagaagccctcaatcaccgcatcgtgcagcaggctaaggagatgacagaaactatggagaagaagttagtagaagaaacgaaacaactggaactcgaccttaatgatgaaaggctgagatatcagaaccttctgaatgagttcagtcgcctggaagaaagatatgatgacctcaaggaagagatgacccttatggtgcatgtgcctaagcctggacacaagagaacagactccacccacagcagcaacgagtctgaatatatctttagctctgaaattgcagaaatggaagacattccatcaaggacagaggaaccaagtgagaagaaggtacctctggacatgtcattgttccttaagctccagaagcgggtcacagagctggagcaggagaagcaggtgatgcaggatgagctggaccgcaaggaggagcaggtgctccgcagcaaggccaaggaagaagaaagaccacaaattagaggtgcagaactggaatatgagtcactcaagcgtcaagaactagaatcagaaaacaaaaaactgaagaatgagctaaatgagttgcgcaaggccctcagtgagaaaagtgccccagaggtgaccgccccaggtgcacctgcctaccgtgtcctcatggagcagctgacctctgtgagcgaggagcttgatgtccgcaaggaggaagtcctcatcttaaggtctcaactggtgagccagaaagaggccatccaacccaaggatgacaagaatacaatgacagattccacaatacttttggaagatgtacaaaaaatgaaagataaaggtgaaatagcacaagcatacattggtttgaaagaaacaaatagatcatctgctctggattaccatgagttgaatgaggatggagagctgtggctggtttatgaagggttaaaacaagccaacaggctcctggaatcccagctgcagtcacagaagaggagccatgagaatgaggccgaggccctccgtggggagatccagagcctgaaggaggagaacaaccgacagcagcagctgctggcccagaacctgcagctgcccccagaggcccgcattgaggccagcctgcagcacgagatcacccggctgaccaacgaaaacttggatttgatggaacaacttgaaaaacaggataagacggtccgtaaactgaaaaaacaactgaaagtatttgccaaaaaaattggcgaactagaagtgggccagatggagaacatatccccaggacagatcattgatgaacccatccgaccagtcaacattcccaggaaagaaaaggatttccaagggatgctggaatacaagaaggaggatgagcaaaaacttgttaagaacctgattctggaactgaagccacgtggtgtagcagtcaatttgattccaggattaccggcatatatcctgttcatgtgtgttcgacatgctgactacctgaatgatgatcagaaagtaaggtcgttgctaacatcaacaattaacagcatcaaaaaagtattgaagaaaagaggtgatgattttgaaaccgtctccttctggctctctaacacatgccgatttttgcactgcttgaaacagtacagtggagaagagggctttatgaagcacaacacatctcgccagaatgaacactgcctcaccaattttgacctggctgagtatcggcaggtgctgagtgacttggccattcagatctaccagcagctcgtgcgggtgttagagaacatccttcagccaatgattgtctcaggcatgctggaacatgaaacgattcagggcgtgtctggggtgaagcccacagggttgagaaagcgaacctccagtatcgccgatgagggcacctacacactggactccatcctccggcagctcaactccttccactcggtcatgtgtcagcatggcatggaccctgaactgatcaagcaggtggtcaagcagatgttctacatcataggggccatcaccctgaacaaccttctcctgcggaaggacatgtgctcctggagtaaaggcatgcagatcaggtacaatgtcagtcaactggaagaatggctgcgtgacaagaatctgatgaatagtggggctaaagaaaccctggaacctctcattcaggctgctcaacttttgcaagtgaaaaagaaaacagatgatgatgcagaagccatttgttctatgtgcaatgctttaactactgcccagattgtgaaagtgttgaatttgtatactccagttaatgagtttgaagaaagagtctctgtgtcgttcattcgtactatacagatgcgtttacgagacaggaaagactctccccagctgctcatggatgctaaacacatctttcctgtcacctttcctttcaacccatcttccctcgcactagaaaccatccagattccagccagcctcggcctgggcttcatttcacgggtctgaaagtgatgtccaggcaaaaattgacaatacatttcttgcccgaaataagaacccattatttccagtgagttactgaaaatacatttttaaagagaaagtactgattatctcccaaatgagaagtcattaactggaaatctccctagaatactttcatcactttggaaacaaagataggctctttcgtgctgtgttatctttatagcaacactcatccttaaccaactaggtaccgtgagtttacatacaggagaatgatggaaggaagggaggaaggaaaggaggagaaaaatgtgtcttcagctggcagcatttattttaaatccttagcactgagtttgaatggtataaaaagtataacttccatagatgagctgttgttaggaaggcaccaaagaacctcctctgcactaaacaggagaatggaaagaaaagtctccattgagtacatatcatgtcagtttagtaatcaattatgttgatattgttaaactggttcaaagaaataaactggcaatatgtaaagtaattcctcatttgtgtcactatgatatagagatattaaaggaatgttggtttgctaaatagtatagatgtccatttgtactatagtttactgagcattttaaattgctgctacatactgtcttcttaaaatgtaagtgatattaggcactacaataagtttctcttgtcaattctgtttacaattcaatcagatcacagttttaactggattatatgcaaatacctacagattcacctgcacaagtagcagacactggaaagtcatgtagtaatatgacaaaatgcttgacatttaggggtaggattagacaaagtggctattgttgatgtcattatttattcaggatgtattacattgatgtgctcattaattttccctgggtggatattgcgtcagggtacagtgttctgtgaagtgacttatttttaactaccagatctgattccttcagtgcatattttcaaccttgacaggttttctctcttcttaatttattaagaattaatctcggctgggcgcggtggctcacgcctgtaatcccagcactttgggaagccaaggtgggcggatcacttcaggttaggagttggagaccagcctggccaacatggcgaaaccctgtctctactaaaaatacaaaaattagccgggcgtggtggcacttgcctgtaatcccagctactcgggaggctgaggcacgagaatcgattaaacctgggaggcggagattgcaatgagatcgaaccactgcactccagcctgggtgacagagagagacactgccttggaaaaaaaaagaatctcactcactatctagagaggattgtcagaatattcacgattcaggtcttgaaactttgattatgcaaaagaaggtatataataaatatttcattatgattcagtttttaaggctttgcagcttctataagtgttctcagatgccactagataattttaaaagcatcatattagaaatactttaagaagacttatataagaaatagaagattgttgaattttacagaggatttggttcattaagacccagattctgtaagttttcattctgaaattctagttaaacatattcaccatttttcttaggaatcttatacaataaatccttcaggttgcacaaaagcaaattattagttttcattagaaactctggttctgaattacaatcataggttatataaatttaactgttagatggtctataaatcttattaaaatatgtgcaatatttatggaagtcaaacagcttcatatcagtgataaagattgttattaaaagataaatactgtctgttaatttacatgggcctcaagttcctcgtttataaaataagagagttggacactgattcttaacatctcctccacatttaaaattctctcttctcagcccttagattctagagagaaaaagctgcagttactcagtaagtccattctctgatggaaagaccagtgtgtagtgcctgtcaattccttaggattaatcaaatgtaaaatcacaagtttgtgtagctgtaacctttcttaaatgtacatgatttatgtacatgcttttagaaggtcctactatatttgtattataattagtttaagtaatttttattacatcatgtattgctttattcagtttgaatacatttatttatttatttgcagtatcaaccagaaacactaccaattgcatcaaattctcccagtttttcctggttgtcaatgcggttttcaatgcacaattaagtcatagccatttggttcgtaccaaatgtgtcagaatctaacagcatccgataggctgtaagttggggagttgctaagaaaatgcaacgtggtacaggctgtccgcctcagccctggaaatctcccagacctcccccagcttcatcctgtgtagcacgactcaacgtgcaccctgaatcttctcaggtcttccaggtcatgctgtagctgtcactgccatgcagcccttttttttactccggacagctcatgtactgaagcgtcatgaaagaaaggctgtggtctgagcccttctctcccatctcctgtctttgtcctgtcaagtgctggagccagagctcctacagctgcccttggtggtttctcctgttcagcgatggtggcacaaaggttctgctattccagggctccagcttcctcccaggtctacccagagctccagatgggggtctgaattaacctctcttggtggcctggagatttttagtcattgacaagaataccttgtaaccagggaaccccaaggcccagtaaatgattctgtataccattttcttgaaggtacaagaagattctgccgactatggggatctttgggccagtttgaggattgctttccctctgaggttctttctctctgtcagccacactttctcacccaacttcagacacaccctgccagcctttcccctactcattcactcttccccttccctcaacttaatcgtctatcccgttgcctgctgtttgactgtgcactgaaggcaggtggatggagtcagtcctcagttgcccctgctggccttcctggtgcttaccatcagcccaatctttgcacagtccttgttgttcttacttctctgcatgcattccttcagaagatcagtcatcaactttttcttaattcctctgtgacacacaatgggaattcaaaggaagagatcttaaaagtcacaacagttctttatcttaataatcccctccccattcaccttactacatgcagactcacctcacacccttacaacttgaagctgaaaatttaaaagtaatttccctttttgcagcttttcctcaggttaaggctttgatctgcctgagagtaactctaaaaggagggaagataaatatgggataaaatccacaaagtgtagcttctaattcctttggaagtttaaaaaatttccacatatctgatgcttcttttgtcaggtgcagaagcacaaaaacatattccgaagccaactgatagggaatttggggattattgtcagtttggagaatttgctgtgttatttcttcatttccatggatagctcatagttggctctttctgggtgagtaattatgtgtaatatagatcaaatcttttactaaggttacagctacatgttaggggaggctatgaaaatactatattattataatttcagtgcagtgattgttgtgagaaataactttcatggtaaccctaggaaaatgggcacctgccaccatcctgagaagtcctcacacaatgccctttctctcttacacacacacacacacacatacacacacacacacccccgtcactaattcatagagttccttagcaggcatagtcaaggatcctctgggtaatgtcagctgcttagtgataaaacagagccaaaactagtgcatcctgttgaaagtaatgcagaaacagtacctgggtccagatatgctttcctgcggcgctttcctctgttacctcgtttcatcctcacagcagcatggacggtaggtggggtcgcttctacaatcatttctgatgatagcttgggaatagagataggggcagtgacttgcctgatgtcgcacagccctctggctgtcctgctttcccatatggagcagtggtggtgtgggcacctgtgatgcaggagactttaaaaatgtcgtgaggtcacgtgctgcccctcctggtacgtgtggaatgcccctggccagcaaggggtgcttttttatcagagttggcagctggcatgtgggaaccgagcaagtgctgcgtaccaagttacttgttttaaggagaccaagtgctcagcgccaggtggttttcttttttgtcatagttacttgctataactcagcttgacttctgtcatgaatcagtgctctctgggaggatgcaatactctgtttgggcattaattggtagcaggttgtctcaaccaaaaagacaggaaacagcaaaagcctctctgaaattaagaggaaagttactctccccacacccatcagagtctttattggagccaccaggtgagctgtgcagcctggacaggcctgcagctataggccaccttcccagtttaggtcctcagcacaggggagcccaagtcactgggtgccttccgagggctgtcactgggcaggccatatacaagtcagtgtgtgcgtgggcactgcagtgtgtgcatgccgtaggtgttgatgggtgctaggaggggtgtcgtgtgcatgcgcgttgaagaggatctgtattgccgtgacctctgttcatggatgagtgcattgtaatttgttctcaggctgtgctgtgagggccgccttaacccttgctcccttcccttctagagctgccttaagttctccagaacttttcttctgtaaaggatatcttgcctggaagggatatcttgccctgtttctcaaggttttgtgagagttttgactggatgtggccctgcatgaccctccttctcctgtacttcctctttcctttccaaatgggaattagaactgtggggcagcaacagtctcagagccagtgagaggccagcttagagaatgcttctgagttagtgggactctgtgtcacaagtaagcaaatgaatatatgaaagaaattatggagataagttagattcttggtaatacttaaatgtcttgctttctactaaccttttgttactaaaggtaaagggtataactcaaactttttgtggacattcttttcaaaattttttaagaaccctgtactataaaaggttgagtaaaaacaggaaagcgtgctataagttcaaatctgttgtattaccctaaattagataaaccaacctgaattatagtagatttctcaatagatgaggaactgaaaaatactatgtaaaatatcttccaaaatgctttttatactttttttatttgtaatttggtctatctaaaatgttcgttagcttaacttaatgggcgttattggattcatatgactaacgtttcctcagtattgtaatgcttgaaatatttgaaagaaaaaatgttgttttttagttgaaactggtatatataattcagtgcttggcaggttagtatatttttatgcatttttcagagtcagcagtttcaaatcttattgttatcatgttataaaattttagcccacatttcaggctccgtaaatcatttgagccattattttttcccaacaaatggtgaattttttctttaaatgtggatatatatgttgtaatttatgattcctggttatgtatttttgtgggatcctgcagtaaaattgacttttttgtgtctttgggagatttaaattgcgctaacagtgttgcgcaaaaatgagttcatgccatttaacatattgtattttaattattaactgtattaatttactatgaaatggacatccttttaactaaaatggaattgaacattgcagttttcaaatatttttccttgttgggtctggaaaaggaattctactttgatctgcatagaaaattttgatacaattttttgaaagttcttaggtgaaacatttacccattaaaaaggaagcagaaatactgagacatgaaaggcattatcaactaactctagactctagaacccattctagcatatctcacgtgcaatttttaaaaataagttaataattcatctcatatcaacaaaagcctttgaaacatgggttttcactagatatcacctagtgctaagataaaaaccaaaacaatatcagaattacatttatgctctaaatttgtagttgtccattgttgtgcttagtaaatgtgtgtcattaatgctgtattctcctagctattatggaaacttgtttaaataaagatatggatataaaga

SEQ ID NO: 22 - DNA, 484 bp, Homo sapiens; identical to SEQ ID NO: 9